(19) United States
(12) Patent Application Publication
(10) Pub. No.: US 2026/0083060 A1
(43) Pub. Date: Mar. 26, 2026

(54) SYSTEMS AND METHODS FOR DETERMINING COTTON QUALITY CHARACTERISTICS AND CLASSIFYING COTTON CLUSTERS

(71) Applicant: Deere & Company, Moline, IL (US)

(72) Inventors: Mahesh SOMAROWTHU, Pune (IN); Noel W. ANDERSON, Fargo, ND (US)

(21) Appl. No.: 18/898,119

(22) Filed: Sep. 26, 2024

(51) Int. Cl. A01D 46/08; G01N 33/00

(52) U.S. Cl. CPC A01D 46/085; G01N 33/0098

(57) ABSTRACT

A cotton harvesting system includes one or more cotton quality characteristic sensors, configured to detect a plurality of cotton quality characteristics and to generate cotton quality characteristic sensor data indicative of the plurality of cotton quality characteristics; one or more processors and memory storing computer executable instructions that, when executed by the one or more processors, cause the one or more processors to performs steps. The steps comprising: obtaining the cotton quality characteristic sensor data; determining, for each cotton cluster of a plurality of cotton clusters generated by a cotton harvester, a respective set of cotton quality metrics based on the cotton quality characteristic sensor data, wherein for each respective set, each cotton quality metric corresponds to a different cotton quality characteristic than the other cotton quality metrics of the respective set; and generating a control signal based on at least one respective set of cotton quality metrics.

### FIELD OF THE DESCRIPTION

The present description relates to mobile agricultural harvesting machines, particularly mobile cotton harvesting machines.

### BACKGROUND

There are a wide variety of different mobile agricultural harvesting machines. Some such mobile agricultural harvesting machines include mobile cotton harvesting machines. Some cotton harvesters, such as cotton pickers and cotton strippers, have a set of row units on the front end of the harvester. The row units act to funnel cotton plants, planted in rows, into the individual row units. In the example of a cotton picker, each row unit has one or more rotatable drums to which a plurality of spindles are mounted. The spindles are rotated to pick seed cotton from the opened cotton bolls entering the row unit. The spindles extend radially from the drum and are supported for rotation about a longitudinal axis of the drum. Each of the spindles is elongate along a longitudinal axis. The spindles also rotate about their longitudinal axis. Rotation of the spindles separates the seed cotton from the cotton plant. Each row unit includes a rotatable doffer that rotates in a counter rotating manner, relative to the rotation of the spindles about the longitudinal axis of the drum, to remove the cotton material from the spindles. The cotton material is then transferred (such as using an air system or other conveying mechanism) into a containment area. On some cotton harvesters, the cotton is transferred from the containment area into a module forming area. Once a module (e.g., a round module) is formed, a door opens at the rearward end of the cotton harvester so that the module can be ejected onto the field.

In the example of a cotton stripper, each row unit has two rotatable stripper rolls to which a combination of one or more brushes and one or more bats are mounted. The stripper rolls, and thus the one or more brushes and one or more bats, are rotated to strip cotton as well as other material from the cotton plant entering the row unit. The material is then transferred using a cross auger to an air system. The air system conveys the material to a cleaning system where cotton is separated from the other stripped material. The separated cotton is then transferred, by the air system, to into a containment area. On some cotton harvesters, the cotton is transferred from the containment area into a module forming area. Once a module (e.g., a cotton round module) is formed, a door opens at the rearward end of the cotton harvester so that the module can be ejected onto the field.

The discussion above is merely provided for general background information and is not intended to be used as an aid in determining the scope of the claimed subject matter.

### SUMMARY

A cotton harvesting system comprising: one or more cotton quality characteristic sensors, on-board a cotton harvester, configured to detect a plurality of cotton quality characteristics and to generate cotton quality characteristic sensor data indicative of the detected plurality of cotton quality characteristics; one or more processors; and memory storing computer executable instructions, executable by the one or more processors, the computer executable instructions, when executed by the one or more processors, causing the one or more processors perform steps comprising: obtaining the cotton quality characteristic sensor data; determining, for each cotton cluster of a plurality of cotton clusters generated by the cotton harvester, a respective set of cotton quality metrics based on the cotton quality characteristic sensor data, wherein, for each respective set, each cotton quality metric corresponds to a different cotton quality characteristic of the plurality of cotton quality characteristics than the other cotton quality metrics of the respective set; and generating a control signal based on at least one respective set of cotton quality metrics.

The cotton harvesting system of any or all previous examples, wherein the one or more cotton quality characteristic sensors comprise one or more of: near infrared (NIR) sensors; terahertz sensors; light sensors; or a high volume instrument sensor system.

The cotton harvesting system of any or all previous examples, wherein the plurality of cotton quality characteristics comprises two or more of: color; fiber length; fiber length uniformity; elongation; contaminants; micronaire; or constituents.

The cotton harvesting system of any or all previous examples, wherein the computer executable instructions, when executed by the one or more processors, further cause the one or more processors to perform a step comprising: providing the respective set of cotton quality metrics for each cotton cluster of the plurality of cotton clusters generated by the cotton harvester to a cotton grouping model to obtain, as a cotton grouping model output, a plurality of cotton groupings, each cotton grouping, of the plurality of cotton groupings, comprising a different set of cotton clusters of the plurality of cotton clusters generated by the cotton harvester.

The cotton harvesting system of any or all previous examples, wherein the cotton grouping model utilizes a K-means clustering algorithm.

The cotton harvesting system of any or all previous examples, wherein the computer executable instructions, when executed by the one or more processors, further cause the one or more processors to perform a step comprising: generating a map, the map including a plurality of cotton cluster indicators, each cotton cluster indicator of the plurality of cotton cluster indicators corresponding to one of the plurality of cotton clusters, located at a location of the map corresponding to a location of the corresponding cotton cluster in a worksite, and visually distinguished to indicate the cotton grouping to which the corresponding cotton cluster belongs.

The cotton harvesting system of any or all previous examples, wherein the map includes a plurality of harvest area indicators, each harvest area indicator of the plurality of harvest area indicators corresponding to one of the plurality of cotton clusters, located at an area of the map corresponding to an area of the worksite from which the cotton, of the corresponding cotton cluster, was harvested.

The cotton harvesting system of any or all previous examples, wherein the map includes a plurality of characteristic indicators, each characteristic indicator indicating a value of a characteristic, different than each of the plurality of cotton quality characteristics, located at an area of the map corresponding to an area of the worksite to which the value of the characteristic corresponds.

The cotton harvesting system of any or all previous examples, wherein the computer executable instructions, when executed by the one or more processors, further cause the one or more processors to perform a step comprising: generating a display, the display including the map and a cotton quality display portion, the cotton quality display portion configured to display the respective set of cotton quality metrics for at least one cotton cluster of the plurality of cotton clusters generated by the cotton harvester.

The cotton harvesting system of any or all previous examples, wherein the computer executable instructions, when executed by the one or more processors, further cause the one or more processors to perform a step comprising: generating a machine assignment assigning a cotton collection and transport machine to a cotton grouping of the plurality of cotton groupings.

The cotton harvesting system of any or all previous examples, wherein the computer executable instructions, when executed by the one or more processors, further cause the one or more processors to perform a step comprising: generating a route for the cotton collection and transport machine based on the machine assignment.

The cotton harvesting system of any or all previous examples, wherein the control signal controls a controllable subsystem of the cotton harvester.

The cotton harvesting system of any or all previous examples, wherein the control signal controls a controllable subsystem of a cotton collection and transport machine.

A computer implemented method comprising: detecting, with one or more cotton quality characteristic sensors on-board a cotton harvester, a plurality of cotton quality characteristics and generating cotton quality characteristic sensor data indicative of the detected plurality of cotton quality characteristics; obtaining the cotton quality characteristic sensor data; determining, for each cotton cluster of a plurality of cotton clusters generated by the cotton harvester, a respective set of cotton quality metrics based on the cotton quality characteristic sensor data, wherein, for each respective set, each cotton quality metric corresponds to a different cotton quality characteristic of the plurality of cotton quality characteristics than the other cotton quality metrics of the respective set; and generating one or more control signals based on at least one respective set of cotton quality metrics.

The computer implemented method of any or all previous examples and further comprising: providing the respective set of cotton quality metrics for each of cotton cluster of the plurality of cotton clusters generated by the cotton harvester to a cotton grouping model to obtain, as a cotton grouping model output, a plurality of cotton groupings, each cotton grouping, of the plurality of groupings, comprising a different set of cotton clusters of the plurality of cotton clusters generated by the cotton harvester.

The computer implemented method of any or all previous examples and further comprising: generating a map, the map including a plurality of cotton cluster indicators, each cotton cluster indicator of the plurality of cotton cluster indicators corresponding to one of the plurality of cotton clusters, located at a location of the map corresponding to a location of the corresponding cotton cluster in a worksite, and visually distinguished to indicate the cotton grouping to which the corresponding cotton cluster belongs.

The computer implemented method of any or all previous examples and further comprising: generating a machine assignment assigning a cotton collection and transport machine to a cotton grouping of the plurality of cotton groupings; and generating a route for the cotton collection and transport machine based on the machine assignment.

The computer implemented method of any or all previous examples, wherein generating the one or more control signals comprises one or more of: generating a control signal to control a controllable subsystem of the cotton harvester; or generating a control signal to control a controllable subsystem of cotton collection and transport machine.

A cotton harvester comprising: one or more cotton quality characteristic sensors configured to detect a plurality of cotton quality characteristics and to generate cotton quality characteristic sensor data indicative of the detected plurality of cotton quality characteristics; one or more processors; and memory storing computer executable instructions, executable by the one or more processors, the computer executable instructions, when executed by the one or more processors, causing the one or more processors perform steps comprising: obtaining the cotton quality characteristic sensor data; determining, for each cotton cluster, of a plurality of cotton clusters generated by the cotton harvester, a respective set of cotton quality metrics based on the cotton quality characteristic sensor data, wherein, for each respective set, each cotton quality metric corresponds to a different cotton quality characteristic of the plurality of cotton quality characteristics than the other cotton quality metrics of the respective set; and generating a control signal based on at least one respective set of cotton quality metrics.

The cotton harvester of any or all previous examples, wherein the computer executable instructions, when executed by the one or more processors, further cause the one or more processors to perform a step comprising: providing the respective set of cotton quality metrics for each cotton cluster of the plurality of cotton clusters generated by the cotton harvester to a cotton grouping model to obtain, as a cotton grouping model output, a plurality of cotton groupings, each cotton grouping, of the plurality of cotton groupings, comprising a different set of cotton clusters of the plurality of cotton clusters generated by the cotton harvester.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

FIG. 1 is a perspective view of one example of a cotton harvester as a cotton stripper.

FIG. 2 is a perspective view of one example of a cotton harvester as a cotton picker.

FIG. 3 is a partial pictorial, partial schematic illustration of one example cotton harvester.

FIG. 4 is a block diagram showing one example of a cotton harvesting system architecture, including, among other things, a cotton harvester in the previous FIGS., in more detail, according to some examples of the present disclosure.

FIG. 5 is a block diagram showing one example of a cotton quality classifier system in more detail.

FIG. 6 is a block diagram showing one example of a logistics system in more detail.

FIG. 7 is a pictorial illustration showing one example display.

FIG. 8 is a pictorial illustration showing one example display.

FIG. 9 is a pictorial illustration showing one example display.

FIG. 10 is a pictorial illustration showing one example display.

FIG. 11 is a flow chart illustrating one example of operation of a cotton harvesting system architecture.

FIG. 12 is a block diagram showing one example of items of a cotton harvesting system architecture in communication with a remote server architecture.

FIGS. 13-15 show examples of mobile devices that can be used in a cotton harvesting system architecture.

FIG. 16 is a block diagram showing one example of a computing environment that can be used in a cotton harvesting system architecture.

### DETAILED DESCRIPTION

For the purposes of promoting an understanding of the principles of the present disclosure, reference will now be made to the examples illustrated in the drawings, and specific language will be used to describe the same. It will nevertheless be understood that no limitation of the scope of the disclosure is intended. Any alterations and further modifications to the described devices, systems, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure.

The price a cotton grower receives for harvested cotton is determined, at least in part, based on the quality of the harvested cotton. In current systems, harvested cotton is often taken to a facility, such as a cotton gin facility or some other facility, where the cotton is tested for quality and the price is determined. The higher the quality, the higher the price. Additionally, the quality of the cotton can also determine its end use. For example, only cotton of a certain quality may be used for high end garment production, and thus, may receive a higher price. Whereas, cotton of a lower quality may be used for other production, including lower end garment production or cloth used in other types of products, and thus, may receive a lower price.

Currently, cotton harvesting systems are limited in functionality to collect and utilize sensor data, generated by sensors on-board a cotton harvester, to determine cotton quality metrics, and thus, it can be difficult for growers to collect and analyze cotton quality data for decision-making and control during a current operation, for future decision-making, to plan logistics of cotton collection and transport, to estimate profitability, as well as for various other decision-making, control, planning, and analysis.

The present description proceeds with example systems and methods that allow for on-board generation of cotton quality data. The cotton quality data can be used for control of one or more machines, operation planning, information presentation, and for various other purposes.

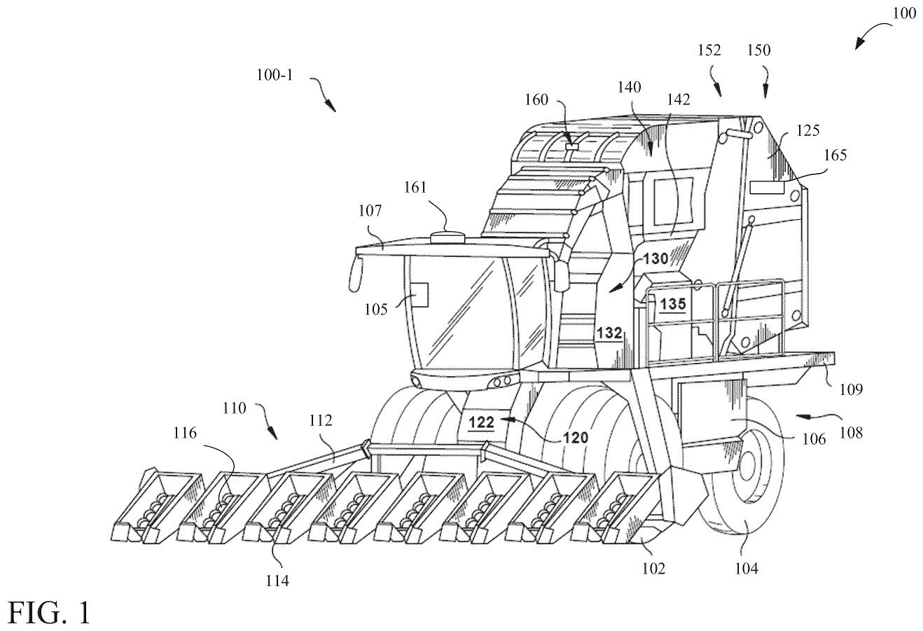

FIG. 1 is a perspective view showing one example of a cotton harvester 100 as a cotton stripper (illustratively 100-1). Cotton stripper 100-1 includes a chassis 109 (e.g., main frame) that is supported by a set of ground engaging traction elements, illustratively shown as front wheels 102 and rear wheels 104, although, in other examples, other types of ground engaging traction elements are contemplated, such as tracks. An operator compartment 107 is supported by the chassis 109 and includes operator interface mechanisms 105. A power plant 108, such as an engine 106, can be supported below the chassis 109. Water, lubricant, and fuel tanks may also be supported on the chassis 109 though are not shown in FIG. 1.

A cotton stripper header 110 includes a frame 112 and is coupled to the chassis 109. As cotton stripper 100-1 moves through a field, cotton stripper header 110 engages rows of cotton plants. The cotton stripper header 110 includes a plurality of cotton stripper heads 114 (e.g., cotton stripper row units) arranged side-by-side across the front of cotton stripper 100-1. Each cotton stripper head 114 may be identical to other stripper heads 114, so the internal structure for one stripper head 114 will be described below with the understanding that the description may also apply to other stripper heads 114. The cotton stripper heads 114 engage rows of crop plants at the field and strip cotton bolls (ripe and unripe) as well as other plant matter from the cotton plants. The cotton stripper heads 114 each include a pair of opposed (e.g., disposed on opposite sides of the stripper head 114 and thus the opposite side of the respective row) stripper rolls 116 that are configured to rotate to strip material from the cotton plants. Each stripper roll 116 can include a combination of one or more bats and one or more brushes disposed about the circumference of each roll and extending along a length of the stripper roll 116. The stripper header 110 can also include a cross auger (not shown) that delivers material stripped by each stripper head 114 towards conveyance system 120 (illustratively shown as an air system).

The cotton stripper 100-1, as illustrated, includes as a conveyance system, an air system 120. Air system 120 can include a crop conveyor component that conveys cotton through the cotton harvester, one or more sensors 160, and a crop conveyor device (e.g., one or more air ducts and an air flow generator). In some examples, the crop conveyer component can include one or more air ducts 122. The air ducts 122 are coupled to, and aligned with header 110, so that the cotton harvested by the header 110 can be transported into the cotton stripper 101 through the air ducts 122 of the air system 120 powered by air flow (e.g., an air flow generated by an air flow generator, such as a fan or blower). In some examples, there is a respective air duct 122 for each stripper head 114.

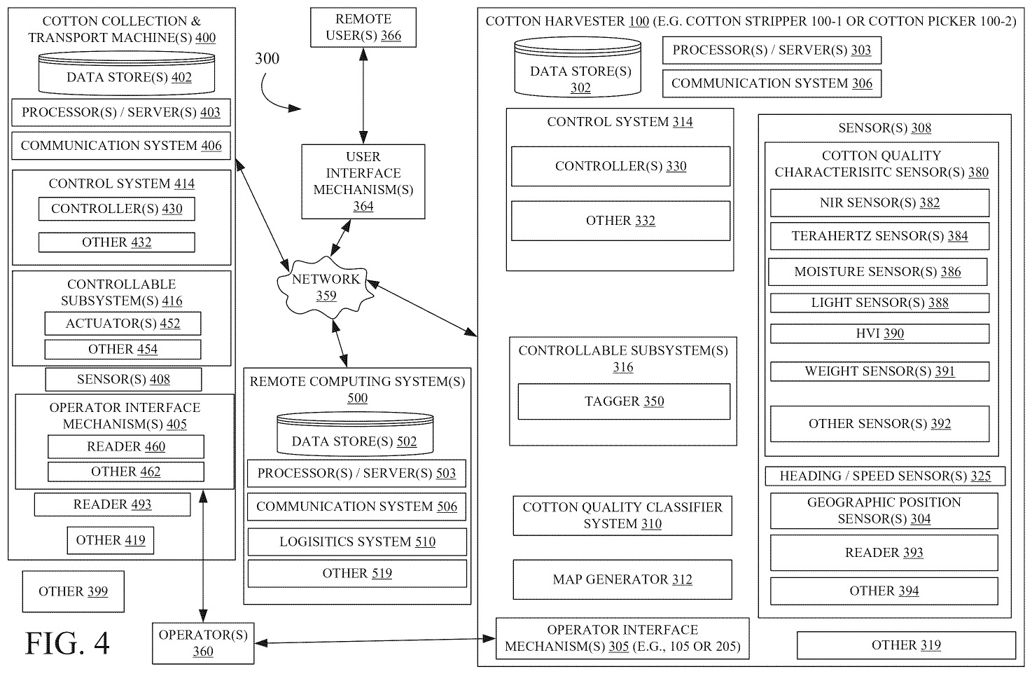

Some of one or more sensors 160 can monitor air flow and/or crop mass flow in the air ducts 122 of the air system 120. In some implementations, some of one or more sensors 160 can be positioned in the air ducts 122. As an example, cotton stripper 101 may include, as some of one or more sensors 160, a plurality of mass flow sensors that are mounted across the width of the air ducts 122. In other examples, one or more mass flow sensors can be positioned adjacent the air ducts 122. In the illustrated example, one or more sensors 160 may include a plurality of mass flow sensors that are mounted behind the air ducts 122. The air flow, and/or crop mass flow, can be monitored using various types of sensors such as, but not limited to, an HDOC yield monitor, a vacuum sensor, an air speed sensor, etc. As an example, the HDOC yield monitor is a microwave-based controller that bounces a signal off a flowing crop to detect a change in velocity with a slowing (or non-existent) crop flow indicative of an air duct 122 being overloaded or plugged. The mass flow information generated by mass flow sensors can be used to derive feedrate or yield. One or more sensors 160 can also include one or more cotton quality characteristic sensors (e.g. 380). Cotton quality characteristic sensors will be shown and described in more detail in FIG. 4. One or more cotton quality characteristic sensors, can be disposed at various locations on cotton stripper 100-1, including locations different than the location shown in FIG. 1. For example, but not by limitation, one or more cotton quality characteristic sensors could be located on header 110, or in cotton receptacle 152 or module builder 150, or accumulator 142, or at each of a combination of locations. In some examples, cotton quality characteristic sensors measure cotton quality characteristics as the cotton continuously moves by the sensors. In some other examples, a sample of the material flow may be captured, held in place during measurement, and then released back into the material flow. In some examples, cotton quality characteristic data may be timestamped or georeferenced using a geographic position sensor 161. Geographic position sensor 161 can be global navigation satellite system (GNSS) receiver or another type of geographic position sensor, some examples of which are described below.

As illustrated in FIG. 1, cotton stripper 100-1 can also include a cleaner system 130. Material travels from the air ducts 122 to the cleaner system 130 and then from the cleaner system 130 to an accumulator system 140. In some examples, the ducts 122 can include a bypass system such that seed cotton, already separated from foreign material can bypass the cleaner system 130 and travel to the accumulator system 140. The foreign material, and cotton interspersed with foreign material, being heavier, will naturally fall to the cleaner system 130. The cleaner system can include a crop cleaner component that cleans the harvested cotton, that is, separates the seed cotton from other material. In some examples, the crop cleaner component can include a cleaner 132 which may include a plurality of components, such as a feeder, doffers, brushes, saw drums, grid bars, a trash auger, ducts, an air generator (e.g., a fan or a blower), as well as various other components. In addition, cotton harvester cotton stripper 100-1 may include load sensors that sense a hydraulic pressure used to drive components of the cleaner system 130 at given speeds. Where the components of the cleaner system 130 are driven by an electric motor, the load sensors may be one or more speed sensors and current sensors.

The cleaned cotton is transported from the cleaner system 130 to the accumulation system 140.

The accumulation system 140 can include a crop accumulator component that temporarily stores the harvested crop and one or more sensors. In some examples, the crop accumulator component can comprise an accumulator 142 and an accumulator capacity monitor. The accumulator 142 is configured to receive cotton harvested by the cotton stripper header 110.

Cotton stripper 100-1 also includes a feeder system 135 that receives cotton from the accumulator 142. The feeder 135 can include a plurality of rollers and motors that compress and transfer the cotton to a cotton receptacle 152 at a feedrate.

The cotton receptacle 152 can include a module builder 150 having one or more bailer belts. The module builder 150 can build a module of cotton, such as cotton round module. In other examples, the cotton stripper 100-1 need not include a module builder, instead, the cotton may be ejected by the air system 120 into an internal hopper and from the internal hopper is transferred from the harvester into an accompanying receptacle (which may be towed by another vehicle). Cotton stripper 100-1 can also include a controllable discharge gate that can be controllably opened and closed to release harvested cotton (e.g., a cotton module, such as a cotton round module). Cotton stripper 100-1 can also include a tagger 165 (such as a QR code tag printer or an RFID tag printer) that prints and applies unique tags and applies them to the modules for purposes, such that IDs and information about each module can be identified. Taggers will be discussed in more detail in FIG. 4.

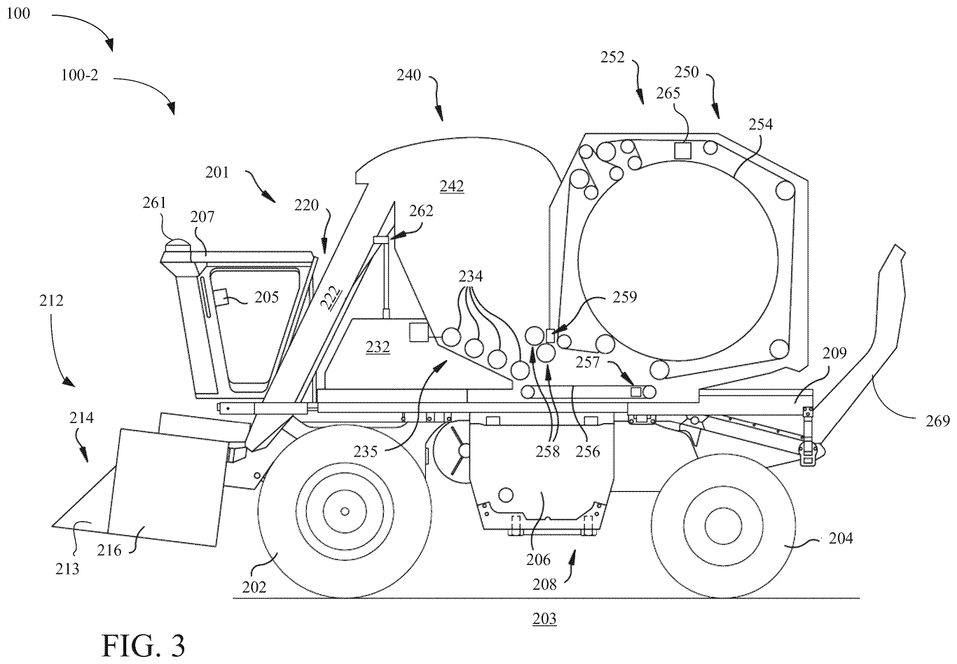

The internal structure and operation of accumulator system 140, feeder system 135, and crop receptacle 152 of cotton stripper can be similar to accumulator system 240, feeder system 235, and crop receptacle 252 of cotton picker 201 which is shown in more detail in FIG. 3. It will be understood that the cotton stripper 100-1 can include various components illustrated and detailed in FIG. 3.

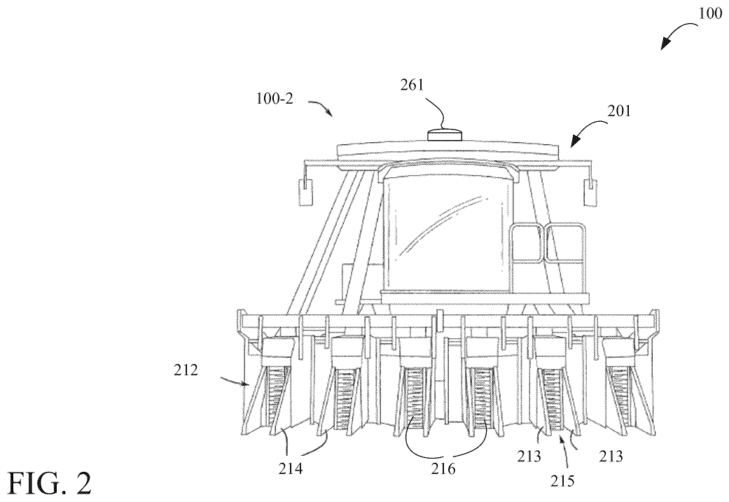

With reference now to FIGS. 2 and 3, which illustrate an example of a cotton harvester 100 as a cotton picker (illustratively 100-2). Cotton picker 100-2 includes a chassis 209 (e.g., main frame) that is supported by a set of ground engaging traction elements, illustratively shown as front wheels 202 and rear wheels 204, although, in other examples, other types of ground engaging traction elements are contemplated, such as tracks. An operator compartment 207 is supported by the chassis 209 and includes operator interface mechanisms 205. A power plant, such as an engine 206, can be supported below the chassis 209. Water, lubricant, and fuel tanks 232 may also be supported on the chassis 209.

A cotton picker header 212 is coupled to the chassis 209. As cotton picker 100-2 moves through a field 203, cotton picker header 212 engages cotton plants. The cotton picker header 212 includes a plurality of cotton picker heads 214 (e.g., cotton picker row units) arranged side-by-side across the front of the cotton picker 201. Each cotton picker head 214 may be identical to the other picker heads 214, so the internal structure for one picker head 214 will be described below with the understanding that the description may also apply to other picker heads 214. Each picker head 214 may include a pair of separators 213 laterally spaced apart from one another and forming a channel 215 disposed between them. The channels 215 receive the rows of cotton plants as the cotton picker 201 is driven through field 203, and, as such, the channels 215 are laterally spaced apart from one another substantially the same distance as the rows of the cotton plants to be picked. Each cotton picker head 214 includes a respective cotton picking unit 216. Cotton picking units 216 remove cotton from the cotton plants.

The cotton picker 100-2, as illustrated, includes as a conveyance system, an air system 220. Air system 220 can include a crop conveyor component that conveys cotton through the cotton picker 100-2, one or more sensors 262, and a crop conveyer device (e.g., one or more air ducts and an air flow generator). In some examples, the crop conveyor component can include one or more air ducts 222. The air ducts 222 are coupled to, and aligned with header 212 so that the cotton harvested by the header 212 can be transported into the cotton picker 100-2 through the air ducts 222 of the air system 220 powered by air flow (e.g., an air flow generated by an air flow generator, such as a fan or blower). In some examples, there is a respective air duct 222 for each picker head 214.

The one or more sensors 262 can monitor air flow and/or crop mass flow in the air ducts 222 of the air system 220. In some implementations, some of one or more sensors 262 can be positioned in the air ducts 222. As an example, one or more sensors 262 may include a plurality of mass flow sensors that are mounted across the width of the air ducts 222. In other examples, some of one or more sensors 262 can be positioned adjacent the air ducts 222. In the illustrated example, one or more sensors 262 may include a plurality of mass flow sensors that are mounted behind the air ducts 222 with one cotton mass flow sensor mounted per row unit. The air flow, and/or crop mass flow, can be monitored using various types of sensors such as, but not limited to, an HDOC yield monitor, a vacuum sensor, an air speed sensor, etc. As an example, the HDOC yield monitor is a microwave-based controller that bounces a signal off a flowing crop to detect a change in velocity with a slowing (or non-existent) crop flow indicative of an air duct 222 being overloaded or plugged. The mass flow information generated by mass flow sensors can be indicative of feedrate or yield. One or more sensors 262 can also include one or more cotton quality characteristic sensors (e.g. 380). Cotton quality characteristic sensors will be shown and described in more detail in FIG. 4. One or more cotton quality characteristic sensors, can be disposed at various locations on cotton picker 100-2, including locations different than the location shown in FIGS. 2-3. For example, but not by limitation, one or more cotton quality characteristic sensors could be located on header 212, or in cotton receptacle 252 or module builder 250, or accumulator 242, or at each of a combination of locations. In some examples, sensors 262 measure cotton quality characteristics as the cotton continuously moves by the sensors. In some other examples, a sample of the material flow may be captured, held in place during measurement, and then released back into the material flow. In some examples, cotton quality characteristic data may be timestamped or georeferenced using a geographic position sensor 261. Geographic position sensor 261 can be global navigation satellite system (GNSS) receiver or another type of geographic position sensor, some examples of which are described below.

In some examples, a crop receptacle 252 is coupled to the air duct system 220. In some examples, the crop receptacle 252 is a module builder 250 having one or more belts 254. As an example, module builder 250 can be used to build a module of the crop, such as a round module of cotton. In other examples, the cotton may not be built into a module, instead the cotton may be transferred as a cluster to another area (e.g., a boll buggy, or onto the worksite to be later retrieved). Cotton picker 100-2 can also include a tagger 265 (such as a QR code label printer or an RFID labels printer) that prints and applies unique labels and applies them to the modules for purposes, such that IDs and information about each module can be identified. Taggers will be discussed in more detail in FIG. 4.

Cotton picker 100-2 can include an accumulator system 240. The accumulator system 240 can include a crop accumulator component that temporarily stores the harvested crop. In some examples, the crop accumulator component can comprise an accumulator 242. The accumulator 242 is configured to receive cotton harvested by the cotton picker header 212.

A feeder 235 is coupled to the chassis 209. The feeder 235 can receive cotton from the accumulator 242. The feeder 235 can include a plurality of meter rollers 234 that compress the cotton and transfer the cotton to the module builder 250 at a feed rate. A first motor 225 is positioned to rotate the plurality of meter rollers 234. The first motor 225 may be hydraulic or electric.

A plurality of beater rollers 258 cooperate with the plurality of meter rollers 234 to transfer the cotton to the module builder 250 at the feed rate. A second motor 259 can be positioned to rotate the plurality of beater rollers 258. The second motor 259 may be hydraulic or electric.

A feeder belt 256 can receive crop from the plurality of meter rollers 234 and beater rollers 258 and transfer the crop to the module builder 250 at the feed rate. A third motor 257 is positioned to rotate the feeder belt 256. The third motor 257 may be hydraulic or electric.

It will be understood that cotton cluster, as used herein, refers to a discrete collection of cotton. In some examples, a cotton cluster can be a module (e.g., a round module, etc.). In other examples, such as where a cotton harvester does not include an on-board module builder, a cotton cluster can refer to a loose (e.g., non-wrapped, etc.) collection of cotton, such as a loose collection stored in an internal hopper, a loose collection transferred to external storage tank, or a loose collection otherwise unloaded (e.g., onto the field) by the harvester in a discrete collection (e.g., pile). Additionally, as used herein, a grouping is a set of one or more cotton clusters.

FIG. 4 is block diagram of a cotton harvesting system architecture 300 (also referred to herein as cotton harvesting system 300 or system 300). As can be seen in FIG. 4, cotton harvesting system 300 includes cotton harvester 100, one or more cotton collection and transport machines 400, one or more remote computing systems 500, one or more user interface mechanism(s) 364, and can include other items 399 as well.

Cotton harvester 100 can be similar to cotton stripper 100-1 or to cotton picker 100-2, or can be another type of cotton harvester. Cotton harvester 100, itself, includes, one or more data stores 302, one or more processors or servers 303, one or more operator interface mechanisms 305, communication system 306, one or more sensors 308, cotton quality classifier system 310, control system 314, one or more controllable subsystems 316, and can include various other items and functionality 319, including, but not limited to, other items and functionality previously described in FIGS. 1-3.

Cotton collection and transport machines 400 are machines that collect and transport cotton harvested by cotton harvester 100, such machines can include tractors with attachments for picking up the cotton, such as attachments for picking up cotton modules, as well as towing vehicles that tow trailers or carts (e.g., cotton buggies, flat bed trailers, etc.) that receive the cotton. Cotton collection and transport machines 400, themselves, include one or more data stores 402, one or more processors or servers 403, one or more operator interface mechanism 405, communication system 406, one or more sensors 408, control system 414, one or more controllable subsystems 416, and can include other items and functionality 419.

Remote computing systems 500 can be any of a variety of remote computing systems, such as mobile devices, a remote network, a farm manager system, a vendor system, or a wide variety of other remote systems. Remote computing systems 500, themselves, include one or more data stores 502, one or more processors or servers 503, a communication system 506, a logistics system 510, and can include other items and functionality 519.

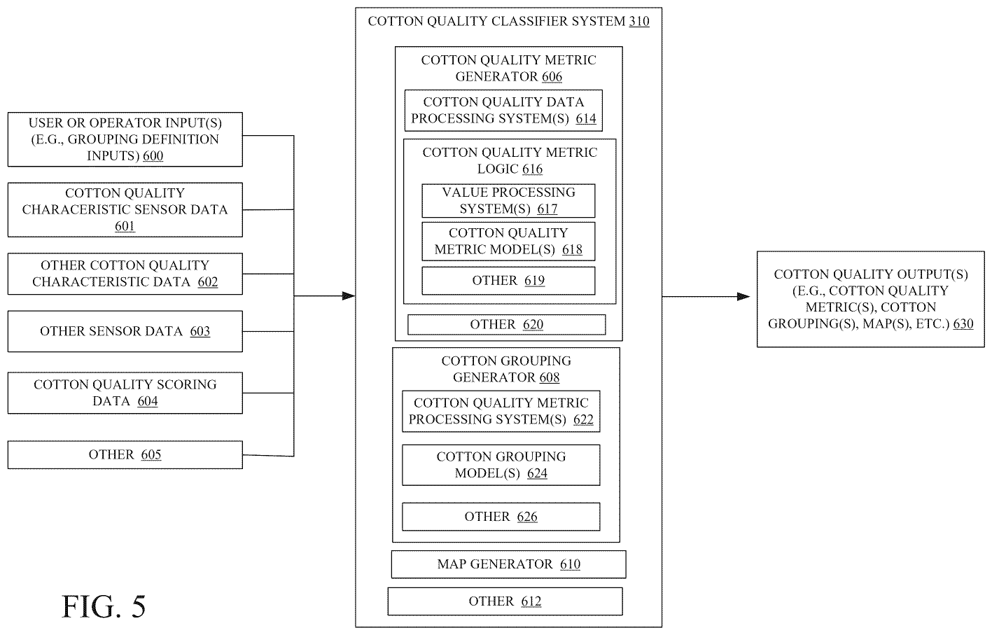

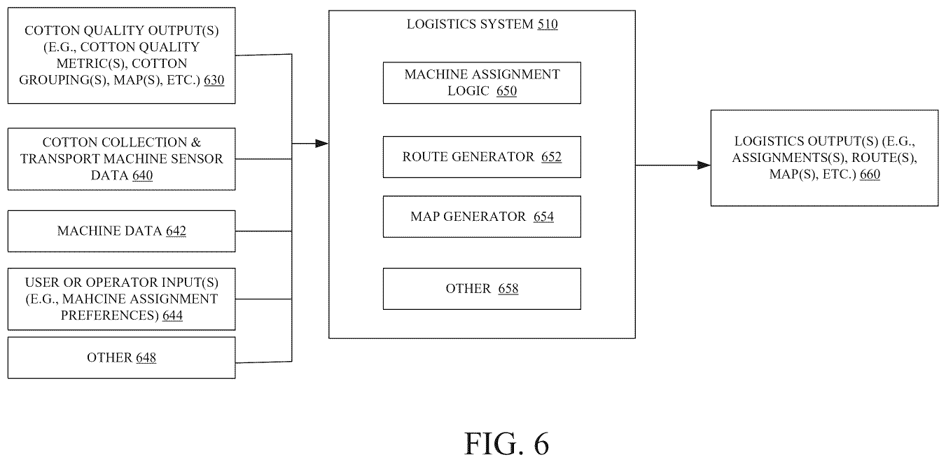

Data stores 302, 402, and 502 store a variety of data. For example, but not by limitation, the data can include worksite data, data generated by other items of system 300, data provided by operators 360 or users 366, historical data, data provided by third-parties, as well as any of a variety of other data. Some examples of the data are shown in FIGS. 5 and 6. Additionally, data store 302 can store, as data, computer executable instructions that are executable by one or more processors or servers 303 to implement other items or functionalities of system 300. Additionally, data store 402 can store, as data, computer executable instructions that are executable by one or more processors or servers 403 to implement other items or functionalities of system 300. Additionally, data store 502 can store, as data, computer executable instructions that are executable by one or more processors or servers 503 to implement other items or functionalities of system 300. It will be understood that data stores 302, 402, and 502 can include different forms of data stores, for instance one or more of volatile data stores (e.g., Random Access Memory (RAM)) and non-volatile data stores (e.g., Read Only Memory (ROM), hard drives, solid state drives, etc.).

Communication system 306 is used to communicate between components of cotton harvester 100 or with other items of system 300, such as machines 400, remote computing systems 500, and user interface mechanisms 364. Communication system 406 is used to communicate between components of a machine 400 or with other items of system 300, such as other machines 400, cotton harvester 100, remote computing systems 500, and user interface mechanisms 364. Communication system 506 is used to communicate between components of a remote computing system 500 or with other items of system 300, such as other remote computing systems 500, cotton harvester 100, machines 400, and user interface mechanisms 364.

Communication systems 306, 406, and 506 can each include one or more of wired communication circuitry and wireless communication circuitry, as well as wired and wireless communication components. In some examples, communication systems 306, 406, and 506 can each be a cellular communication system, a system for communicating over a wide area network or a local area network, a system for communicating over a controller area network (CAN), such as a CAN bus, a system for communication over a near field communication network, or a communication system configured to communicate over any of a variety of other networks, or any combination of such systems. Communication systems 306, 406, and 506 can each also include a system that facilitates downloads or transfers of information to and from a secure digital (SD) card or a universal serial bus (USB) card, or both. Communication systems 206 and 306 can each utilize network 359. Networks 359 can be any of a wide variety of different types of networks such as the Internet, a cellular network, a wide area network (WAN), a local area network (LAN), a controller area network (CAN), a near-field communication network, or any of a wide variety of other networks, or any combination of such networks.

Sensors 308 can include one or more cotton quality characteristic sensors 380, heading/speed sensors 325, geographic position sensors 304, reader 393, as well as other sensors previously described herein. Cotton quality characteristic sensors 380 illustratively detect one or more cotton quality characteristics and generate sensor data (e.g., signals, images, etc.) indicative of the detected one or more cotton quality characteristics. Cotton quality characteristics can include color, fiber length, fiber length uniformity, elongation, contaminants (e.g., trash and extraneous matter), micronaire, constituents (e.g., the concentration of constituents such as moisture, seed oil, seed protein, seed gossypol, etc.), weight, moisture, as well as other characteristics. Cotton quality characteristic sensors 380 can include one or more near infrared (NIR) sensors 382, one or more terahertz sensors 384, one or more moisture sensors 386, one or more light sensors 388, one or more high volume instrument (HVI) sensors 390, weight sensors 391, and can include a variety of other sensors 392 that detect cotton quality characteristics. 2

NIR sensors 382 illustratively include a transmitter that transmits near infrared (NIR) light towards a sample (cotton) and a detector that captures the reflected light to detect quality characteristics of the sample (cotton). The difference in intensity of the reflected light from that of the transmitted light can be indicative of, and can thus be used to detect, characteristics of the cotton. In some examples, NIR signal absorption/transmission may be measured.

Terahertz sensors 384 illustratively include a transmitter that transmits electromagnetic (EM) radiation in the terahertz band (as used herein a frequency between 0.1 terahertz and 30 terahertz) towards a sample (cotton) and a detector that captures the EM radiation reflected from or attenuated by the sample (cotton) to detect quality characteristic of the sample (cotton). The difference in intensity of the attenuated or reflected EM radiation from that of the transmitted EM radiation can be indicative of, and can thus be used to detect, characteristics of the cotton. In some examples, terahertz signal absorption/transmission may be measured.

Moisture sensors 386 illustratively detect moisture content of the cotton. In some examples, previously NIR sensors 382 or terahertz sensors 384, or both, can be used to detect moisture of the cotton. Thus, while moisture sensors 386 are shown as separate from NIR sensors 382 or terahertz sensors 384, in some examples, moisture of the cotton is detected using data generated by NIR sensors 382 or terahertz sensors 384, or both. In other examples, such as where a cotton harvester 100 does not include NIR sensors 382 or terahertz sensors 384, or even when a cotton harvester 100 does include NIR sensors 382 or terahertz sensors 384, moisture sensors 386 can be separate sensors that do not utilize data generated by NIR sensors 382 or terahertz sensors 384. One example of a separate moisture sensor 386 is a capacitance moisture sensor (e.g., plate capacitor, capacitor probe, etc.) that detects a change in capacitance caused by moisture of a sample (cotton), that is, measures a dielectric constant of the sample (cotton).

Light sensors 388 can include one or more of a variety of different types of sensors. For example, light sensors 388 can include visible light sensors, such as cameras, that image a sample (cotton) to detect quality characteristics of the sample (cotton). Additionally, or alternatively, light sensors 388 can include ultraviolet (UV) light sensors that include a UV light transmitter that transmits UV light towards a sample and a detector that captures the EM radiation reflected from the sample or transmitted through the sample. The difference in intensity of the attenuated or reflected EM radiation from that of the transmitted EM radiation can be indicative of, and can thus be used to detect, quality characteristics of the cotton. 2

A High Volume Instrument (HVI) sensor system 390 includes a plurality of different types of sensors that detect cotton quality characteristics, for example, cameras, colorimeters, moisture sensors, NIR sensors, light sensors, fiber strength sensors, pressure sensors (e.g., for micronaire measurement), other optical or EM radiation sensors, as well as other types of sensors. Those skilled in the art will be familiar with the structure and operation of a HVI sensor system. In some examples, as previously described, samples of the cotton from the cotton flow can be captured, held in place during measurement, such as during measurement by the HVI sensor system 390, and then released back into the material flow.

Geographic position sensors 304 illustratively sense or detect the geographic position or location of cotton harvester 100. Geographic position sensors 304 can include, but are not limited to, a global navigation satellite system (GNSS) receiver that receives signals from a GNSS satellite transmitter. Geographic position sensors 304 can also include a real-time kinematic (RTK) component that is configured to enhance the precision of position data derived from the GNSS signal. Geographic position sensors 304 can include a dead reckoning system, a cellular triangulation system, or any of a variety of other geographic position sensors.

Weight sensors 391 can include any of a variety of sensors that detect a weight of a cotton module (or cotton load), such as strain gauges, load cells, pressure sensors, or other types of sensors. In one example, weight sensors 391 may be associated with discharge arrangement 269 and can generate a weight signal indicative of a weight of a module resting on the discharge arrangement 269.

Heading/speed sensors 325 detect a heading characteristic (e.g., travel direction) or speed characteristics (e.g., travel speed, acceleration, deceleration, etc.), or both, of cotton harvester 100. This can include sensors that sense the movement (e.g., rotation) of ground engaging traction elements or movement of components coupled to the ground engaging traction elements or other elements, or can utilize signals received from other sources, such as geographic position sensors 303. Thus, while heading/speed sensors 325 as described herein are shown as separate from geographic position sensors 304, in some examples, machine heading/speed is derived from signals received from geographic position sensors 304 and subsequent processing. In other examples, heading/speed sensors 325 are separate sensors and do not utilize signals received from other sources.

Reader 393 can be an RFID reader or a QR code reader. In some examples, the cotton module is wrapped and the wrapping material includes an RFID label or a QR code label. The reader 393 can read the label, to uniquely identify the module and associate the location of the module (i.e., location where module is placed in the field as derived from data generated by geographic position sensor 303) as well as detected characteristics of the module (as derived from data generated by cotton quality characteristic sensors 380 and output from cotton quality classifier system 310) with the unique identity. Thus, each module can be uniquely identified, geotagged, and indexed with corresponding quality characteristic data. Later, the label can be read by another reader (e.g., 460, 493, etc.) to verify the identity of the module and to observe the associated quality characteristics. As shown in FIG. 4, it can be seen that a cotton collection and transport machine 400 can include a reader 493. Reader 493 can be similar to reader 393.

As previously discussed, cotton harvester 100 can include one or more of a variety of other sensors 394 that detect a variety of other characteristics, including, but not limited, other sensors previously described herein.

Sensors 408 detect characteristics of cotton collection & transport machines 400 or characteristics around cotton collection & transport machines 400. Sensors 408 can include machine/heading sensors (similar to machine/heading sensors 325) that detect a heading characteristic (e.g., travel direction) or speed characteristics (e.g., travel speed, acceleration, deceleration, etc.), or both, of cotton collection & transport machines 400. Sensors 408 can include geographic position sensors (similar to geographic position sensors 304) that illustratively sense or detect the geographic position or location of cotton collection & transport machines 400.

Sensors 408 can include various other sensors that detect various other characteristics of cotton collection & transport machines 400 or around cotton collection & transport machines 400, or both. It will also be understood that reader 493 is a sensor 408.

Cotton quality classifier system 310 illustratively determines cotton quality metrics for the cotton modules, identifies groups of similar modules, and generates maps that map locations and characteristics of the cotton modules. Cotton quality classifier system 310 will be discussed in greater detail in FIG. 5.

Controllable subsystems 316 can include tagger 350.

Tagger 350 illustratively prints a label (e.g., RFID labels, QR code labels, etc.) for each module of cotton and applies the label to the cotton module. Tagger 350 can include a printer, such as an RFID label printer or a QR code printer. An RFID tag or label, may store a unique identifier identifying the cotton module and other data, including time of production, harvest location, cotton quality characteristic data, as well as other data. In other examples, certain data (e.g., cotton quality characteristic data, etc.) may be stored elsewhere (e.g., remote data store) and associated with the unique identifier. Thus, when the unique identifier is obtained by the RFID reader, the associated data can be retrieved and displayed. A QR code acts as a unique identifier and encodes data which, when the QR code is scanned, can be translated into a human-readable form.

In some examples, a cotton harvester 100 need not include a tagger 350. Instead, the module wrapping material can be embedded with or otherwise have attached thereto, labels (e.g., RFID labels/tags, QR code labels, etc.). The label could be read by reader 393 during or after the formation of each module, such that the label of each module can be stored and associated with data such as a unique identifier identifying the cotton module and other data, including time of production, harvest location, cotton quality characteristic data, as well as other data . . .

Control system 314 can include one or more controllers 330 and can include various other items 332. Controllers 334 illustratively generate control signals to control one or more items of cotton harvester 100. For example, one or more of controllers 330 can generate control signals to control controllable subsystems 316, such as tagger 350. Additionally, one or more of controllers 330 can generate control signals to control communication system 306. Further, one or more controllers 330 can generate control signals to control operator interface mechanisms 305, such as to generate an indication (e.g., display, audible output, haptic output, alert, etc.). In some examples, controllers 330 can generate control signals to control one or more items of cotton harvester 100 based on cotton quality characteristic sensor data generated by cotton quality characteristics sensors 380 or based on an output of cotton quality classifier system 310, or both.

Controllable subsystems 416 can include one or more actuators 452 and can include other items 454.

Actuators 452 are controllable to activate or deactivate components (or functionality) of a cotton collection and transport machine 400 or to adjust operation of a cotton collection and transport machine 400 or of different components (or functionality) of a cotton collection and transport machine 400, or both. Actuators 452 can include any of a variety of different types of actuators, such as hydraulic actuators, pneumatic actuators, electrical actuators, electromechanical actuators, as well as various other actuators. Actuators 452 can include engines, motors, pumps, as well as various other mechanisms. As previously discussed, actuators 452 can be controlled to adjust operation of different components of a cotton collection and transport machine 400, such as the operating speed (e.g., speed of rotation, etc.) of different components of a cotton collection and transport machine 400, direction of movement (e.g., direction of rotation, etc.), of different components of cotton collection and transport machine 400, position (e.g., height above ground, depth into ground, position relative to another component of the mobile work machine, etc.) of different components of a cotton collection and transport machine 400, orientation (e.g., roll, pitch, yaw) of different components of a cotton collection and transport machine 400, as well as various other operating parameters of different components of a cotton collection and transport machine 400. Similarly, actuators 452 can be controlled to adjust operation of a cotton collection and transport machine 400, itself, such as adjusting the travel speed of a cotton collection and transport machine 400 or adjusting a travel direction (heading) of a cotton collection and transport machine 400.

Control system 414 can include one or more controllers 430 and can include various other items 432. Controllers 434 illustratively generate control signals to control one or more items of a cotton collection and transport machine 400. For example, one or more of controllers 430 can generate control signals to control controllable subsystems 416. Additionally, one or more of controllers 430 can generate control signals to control communication system 406. Further, one or more controllers 430 can generate control signals to control operator interface mechanisms 405, such as to generate an indication (e.g., display, audible output, haptic output, alert, etc.). In some examples, controllers 430 can generate control signals to control one or more items of a cotton collection and transport machine 400 based on cotton quality characteristic sensor data generated by cotton quality characteristics sensors 380 or based on an output of cotton quality classifier system 310, or both. Additionally, in some examples, controllers 430 can generate control signals to control one or more items of a cotton collection and transport machine 400 based on an output of logistics system 510.

Logistics system 510 illustratively determines routes and machine assignments for cotton collection and transport machines. 400. Logistics system 400 will be discussed in greater detail in FIG. 6.

FIG. 4 also shows that one or more operators 360 may operate cotton harvester 100 or a cotton collection and transport machine 400, and interact with other items of system 300, such as remote computing systems 500, or user interface mechanisms 364, through operator interface mechanisms (e.g., 305 or 405). In some examples, operator interface mechanisms 305 and 405 may include joysticks, levers, a steering wheel, linkages, pedals, buttons, wireless devices (e.g., mobile computing devices, etc.), dials, keypads, an interface display device (e.g., display screen), actuatable elements (such as icons, buttons, etc.) on a interface display device, a microphone and speaker (where speech recognition and speech synthesis are provided), among a wide variety of other types of control devices. Where a touch sensitive display system is provided, the operators 360 may interact with operator interface mechanisms using touch gestures. These examples described above are provided as illustrative examples and are not intended to limit the scope of the present disclosure. Consequently, other types of operator interface mechanisms may be used and are within the scope of the present disclosure.

In the example of FIG. 4, it can be seen that operator interface mechanisms 405 can also include a reader 460 and other items 462. Reader 460 may be a mobile device useable to read labels of cotton modules. Reader 460 can be an RFID reader or a QR code reader.

FIG. 4 also shows remote users 366 interacting with cotton harvester 100, cotton collection and transport machines 400, and remote computing systems 500, through user interfaces mechanisms 364 over networks 359. In some examples, user interface mechanisms 364 may include joysticks, levers, a steering wheel, linkages, pedals, buttons, wireless devices (e.g., mobile computing devices, etc.), dials, keypads, an interface display device (e.g., display screen), actuatable elements (such as icons, buttons, etc.) on a interface display device, a microphone and speaker (where speech recognition and speech synthesis are provided), among a wide variety of other types of control devices. Where a touch sensitive display system is provided, the users 366 may interact with user interface mechanisms using touch gestures. These examples described above are provided as illustrative examples and are not intended to limit the scope of the present disclosure. Consequently, other types of user interface mechanisms may be used and are within the scope of the present disclosure.

While the example shown in FIG. 4 illustrates items being distributed across system 300 in a particular way, in other examples, one or more of the items shown in FIG. 4 can be, alternatively or additionally, located elsewhere or can be distributed across multiple locations. For example, cotton quality classifier system 310 can, alternatively or additionally, be located at one or more remote computing systems 500. Similarly, logistics system 510 can, alternatively or additionally, be located at cotton harvester 100 or at one or more cotton collection and transport machines 400. Thus, it will be understood that the items in system 300 can be distributed in various ways, including ways that differ from the example shown in FIG. 4.

FIG. 5 is a block diagram of portions of system 300 including cotton quality classifier system 310, shown in FIG. 4, in more detail. FIG. 5 also shows the information flow among the various components shown. As illustrated, cotton quality classifier system 310 obtains (e.g., retrieves or receives) one or more of data 600, 601, 602, 603. 604, and 605 and generates outputs 630 based thereon. The one or more outputs 630, which can include one or more selected cotton quality metrics, cotton groupings, maps, as well as various other items, can be provided to other items of system 300 and can be used to control various items of system 300.

Data 600, 601, 602, 603, 604, and 605 can be stored at one or more of data stores 304, 404, and 504, or at other data stores. The location at which data 601, 602, 603, 604, and 605 are stored may depend on the location at which cotton quality classifier system 310 is located.

User or operator inputs 600 are inputs by provided by an operator 360 or user 366, such as through operator interface mechanisms 305 or user interface mechanisms 364, respectively. In one example, user or operator inputs 600 provide information useable to define cotton quality or cotton groupings, or both. For example, user or operator inputs 600 can include information providing the characteristics, or the weight of characteristics, or both, that are to be taken into account when determining cotton quality. Additionally, or alternatively, user or operator inputs 600 can include information providing the categories by which the groupings are to be classified. Thus, in one example, cotton grouping generator 606 can utilize user or operator inputs in identifying cotton groupings, as will be discussed in further detail below.

Cotton quality characteristic sensor data 601 are sensor data (e.g., sensor signals, images, etc.) generated by cotton quality characteristic sensors 380.

Other cotton quality characteristic data 602 are data indicative of cotton quality characteristics obtained from sources other than cotton quality characteristic sensors 380. It will be understood that, in some examples, at least some cotton quality characteristics may be accounted for which are not detected by sensors 380. For instance, certain genotype data relative to the cotton may be obtained from sources other than sensors 380, such as cotton seed providers (e.g., cotton seed manufacturers or sellers). The genotype data may indicate, among other things, the particular hybrid or cultivar of the cotton. For instance, whether the particular cotton is a pest resistant hybrid or cultivar may weigh into the quality of the cotton.

Other sensor data 603 are sensor data (e.g., sensor signals, images, etc.) generated by sensors other than sensors 380, such as heading/speed sensors 325, geographic position sensor 304, reader 393, or other sensors 394.

Cotton quality scoring data 604 are data useable to generate cotton quality metrics, such as metrics of individual cotton quality characteristics or an overall cotton quality metric, or both. For example, cotton quality scoring data 604 can include lookup tables, equations or functions, expert knowledge (e.g., industry standard scoring methodologies, such as the U.S. Department of Agriculture (USDA) cotton classing standard (American Marketing Service (AMS) Cotton & Tobacco (C&T) classing), etc.), as well as various other data that may be used to generate cotton quality metrics from cotton quality characteristic data (e.g., 601 or 602, or both), for example, to convert values indicated by cotton quality characteristic data (e.g., 601 or 602, or both) to cotton quality metric values.

As shown in FIG. 5, cotton quality classifier system 310 can obtain or utilize various other data 605, including, but not limited to, other data described herein. In one example, other data can include maps of the worksite, the maps of the worksite can indicate or include values of various characteristics (e.g., crop characteristics, terrain characteristics, etc.). The maps may be generated prior to the cotton harvesting operation, such as based on overhead imagery, based on sensor data collected during prior operations, user/operator input, as well as generated in various other ways.

As illustrated in FIG. 5, cotton quality classifier system 310 includes cotton quality metric generator 606, cotton grouping generator 608, map generator 610, and can include other items and functionality 612 as well. Cotton quality metric generator 606, itself, includes one or more cotton quality data processing systems 614, cotton quality metric logic 616, and can include other items as well 618. Cotton quality metric logic 616, itself, can include one or more value processing systems 617, one or more cotton quality metric models 618, and can include other items 619, as well. Cotton grouping generator 608, itself, includes cotton quality metric processing systems 622, one or more cotton grouping models 624, and can include other items 626 as well.

Cotton quality metric generator 606 illustratively processes cotton quality data (e.g., 601 or 602, or both) and generates one or more cotton quality metrics, for example, a metric corresponding to each one of a plurality of cotton quality characteristics or an overall cotton quality metric, or both. Cotton quality data processing systems 614 can include sensor signal processing, image processing, or various other data processing functionalities. Cotton quality data processing systems 614 can extract values from input data, such as values from sensor data (e.g., signals, images, etc.) or values from other input data (e.g., data provided by seed manufacturer or producer). For instance, processing systems 614 can include processing functionality to process a sensor signal to extract a current (electrical current) value, such as milliamps. In another example, processing systems 614 can include processing functionality to process sensor data, such as an image or a sensor signal, to extract a reflectance value, a fluorescence value, a color value, or a pixel value. These are merely some examples.

Cotton quality metric logic 616 generates one or more cotton quality metrics based on the outputs (extracted values) from cotton quality data processing systems 614. For instance, in some examples, cotton quality metric logic 616 may utilize cotton quality scoring data 604. In other examples, cotton quality metric logic 616 may utilize one or more cotton quality metric models 618.

For example, value processing systems 617 determine a corresponding cotton quality metric value for each cotton quality characteristic utilizing the corresponding extracted value output by cotton quality data processing systems 614 and cotton quality scoring data 604. For example, value processing systems 617 may provide, for each of a plurality of a cotton quality characteristics, an extracted value as an input to cotton quality scoring data 604 (e.g., lookup tables, equations or functions, expert knowledge, etc.) to obtain, as an output, a cotton quality metric value corresponding to the characteristic. In other examples, value processing systems 617 may provide, for each of a plurality of cotton quality characteristics, an extracted value as an input to a cotton quality metric model 618 to obtain, as an output, a cotton quality metric corresponding to the characteristic. Cotton quality metric models 618 can be machine learning models, such as a neural network or any other of a number of machine learning models. The cotton quality metric models 618 can be trained based upon historical extracted values and historical cotton quality metrics (such as those obtained from a cotton purchaser or cotton grader, or otherwise provided such as by users or operators). For each cotton module, or each cluster of cotton, generated by harvester 100, cotton quality metric generator 606 can output one or more cotton quality metric values (one for each of a plurality of cotton quality characteristics). In some examples, cotton quality metric logic could generate an overall cotton quality metric based on an aggregation of a plurality of cotton quality metric values (each cotton quality metric value corresponding to a different cotton quality characteristic) or based on an aggregation of a plurality of extracted values (each extracted value corresponding to a different cotton quality characteristic). In some examples, the aggregation May include weighting. The overall cotton quality metric could be a cotton grade, or another type of metric.

Cotton grouping generator 608 generates one or more cotton groupings, each grouping consisting of one or more cotton modules or other cotton clusters generated by harvester 100. Each grouping consists of similar cotton modules or other cotton clusters. In some examples, cotton grouping generator 608 identifies the groupings based, at least in part, on user or operator inputs 600 defining, or providing, the one or more cotton quality characteristics that are to be used in identifying the groupings. For example, cotton quality metric processing systems 622 can provide cotton quality metrics values to a cotton grouping model 624 which provides, as an output, groupings of the cotton modules or other cotton clusters. In one example, a cotton grouping model is an machine learning model, such as a K-means clustering algorithm. In one example, the K-means clustering algorithm may utilize one or more cotton quality characteristics (as classification categories and the output cotton quality metric values for each of the one or more cotton quality characteristics for the cotton to be grouped. The one or more cotton quality characteristics can be defined by user or operator input 600, may be default, or may be provided in another way. Additionally, the number of groupings can also be defined by user or operator input 600, may be default, or may be provided in another way. A K-means clustering algorithm is just one example.

In other examples, cotton grouping models 624 can comprise other types of machine learning models, such as neural networks or any of a variety of the types of machine learning models. In one example, a cotton grouping model 624 may be used to regress a plurality of input values to an output value based on model parameters (such as weights, biases, basis vectors, or other types of parameters). Each input value may be a corresponding cotton quality metric value for a cotton quality characteristic of a module or other cotton cluster. The output value may be a cotton grade, price per pound, or a deduction, points per pound, or some other value. The model parameters can be preset, such as by a user or operator, or some other source, or can be learned. For example, the model parameters may be trained based upon historical cotton quality metric values and historical output values (e.g., historical prices, historical deductions, points per pound, or other historical output values). Thus, each cotton module or other cotton cluster may be assigned an output value, and the output values for each cotton module or other cotton cluster in the plurality of cotton modules or other cotton clusters to be grouped can be provided to another cotton grouping model 624, such as a clustering algorithm (e.g., K-means clustering algorithm) to identify the cotton groupings.

In yet another example, a machine learning model, such as a neural network, can be trained to output cotton groupings. In such an example, the model may receive, as inputs, cotton quality metric values for each of a plurality of cotton quality characteristics for each module or other cotton cluster under consideration, and output cotton groupings. The model can be trained on training data consisting of verified (e.g., historical) cotton quality metric values for each of a plurality of cotton quality characteristics for each module or other cotton cluster in the training data and based on verified cotton groupings corresponding to the modules or other cotton clusters in the training data. The model will iteratively repeat processing on the training data (adjusting model parameters (e.g. weights and biases)) until convergence (or at least until the accuracy is deemed acceptable).

These are merely some examples of how cotton groupings can be identified.

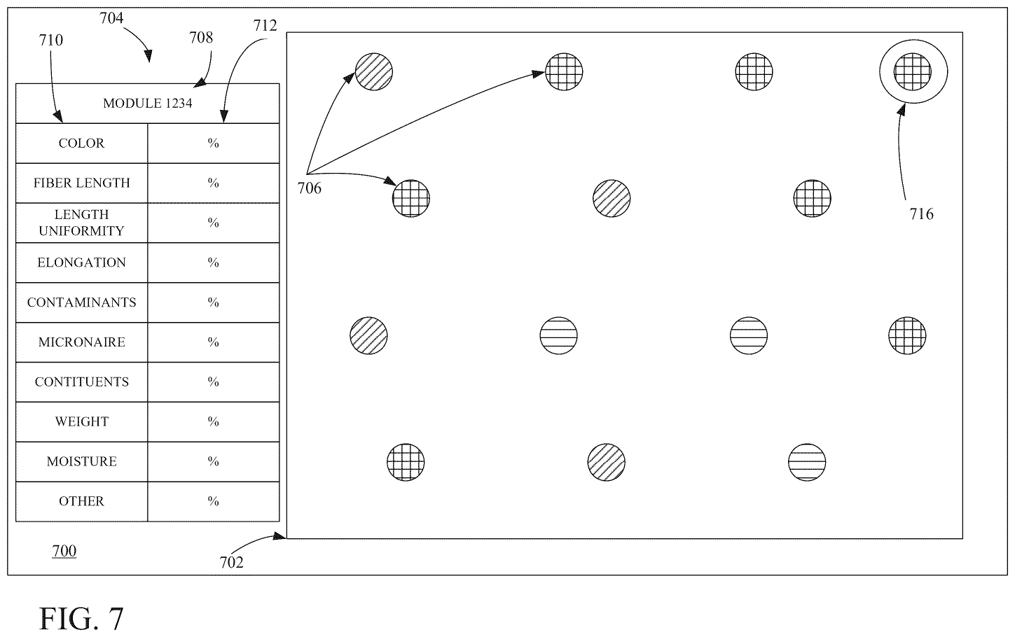

Map generator 610 illustratively generates cotton quality maps, such as a cotton quality map of worksite (e.g., field), that includes indicators (e.g., icons, etc.) showing the location of cotton modules or other cotton clusters at the worksite, and indicates the cotton quality metric values or grouping, or both, for each module or other cotton cluster. In some of examples, the map shows the region of the field harvested to make the cotton cluster, as well, in some examples, other characteristics of the field, such as various crop characteristic, terrain characteristics, as well as various other characteristics relative to the worksite. The locations can be derived from sensor data generated by geographic position sensor 304 as well, in some examples, heading data generated by heading/speed sensors 325. For example, the heading and location of the harvester 100 at the time the module is released (e.g., based on actuation of discharge gate 267 and discharge arrangement 269). In one example, the icon may be a module (e.g., round module) icon or some other icon to indicate an individual module or other cluster of cotton placed at a location in the map corresponding to the location of the individual module or other cotton cluster in the worksite. Additionally, each icon can be visibly marked (e.g. colored, patterned, etc.) to indicate a value or a grouping. For example, the icons of modules or other cotton clusters belonging to the same grouping may be colored or patterned the same. The cotton quality metric values can be displayed next to or over the icons or the icons can be interactable, by a user or operator, such as by touch input or other form of input, which may cause display of a table or other set display of cotton quality metric values for the corresponding module or other cotton cluster. The maps generated by map generator 610 may be provided to other items of system 300 and/or may be presented to an operator or user, or both, via interface mechanisms. One example of a map generated by map generator 610 is shown in FIG. 7.

As can be seen, cotton quality classifier system 310 is operable to generate one or more cotton quality outputs 630. The one or more cotton quality outputs 630 can include one or more cotton quality metrics (e.g., one or more cotton quality metric values), one or more cotton groupings, or one or more cotton quality maps, or any combination thereof. The one or more cotton quality outputs 630 can be provided to other items of system 300 and can be used in the control of one or more mobile machines (e.g., 100 or 400, or both), The one or more cotton quality outputs 630 can be presented to an operator or user (e.g., 360 or 366), or both, via a corresponding interface mechanism (e.g., 305/405 or 364, or a combination thereof).

FIG. 6 is a block diagram of portions of system 300 including logistics system 510, shown in FIG. 4, in more detail. FIG. 6 also shows the information flow among the various components shown. As illustrated, logistics system 510 obtains (e.g., retrieves or receives) one or more of data 630, 640, 642, 644, and 648 and generates outputs 660 based thereon. The one or more outputs 660, which can include one or more machine assignments, routes, maps, as well as various other items, can be provided to other items of system 300 and can be used to control various items of system 300.

Data 630, 640, 642, 644, and 648 can be stored at one or more of data stores 304, 404, and 504, or at other data stores. The location at which data 630, 640, 642, 644, and 648 are stored may depend on the location at which logistics system 510 is located.

Data (or cotton quality outputs) 630 were discussed previously.

Cotton collection & transport machine sensor data 640 are sensor data (e.g., sensor signals, images, etc.) generated by sensors 408, such as heading/speed sensors, geographic position sensors, or other sensors. Sensor data 640 may indicate one or more of a current location of each of a plurality of cotton collection & transport machines 400, a current heading of each of a plurality of cotton collection & transport machines 400, a current speed of each of a plurality of cotton collection & transport machines 400, and various other sensed characteristics of each of a plurality of cotton collection & transport machines 400 or around each of a plurality of cotton collection & transport machines 400.

Machine data 642 are data indicative of characteristics of cotton collection & transport machines 400, such as machine dimensions, carrying capacities, performance capabilities (e.g., machine ratings), machine type/machine model, as well as various other characteristics.

User or operator inputs 644 are inputs by provided by an operator 360 or user 366, such as through operator interface mechanisms 305 or user interface mechanisms 364, 13 respectively. In one example, user or operator inputs 644 provide information useable to determine machine assignments. For example, user or operator inputs 644 can include information providing user or operator preferences, or both, that are to be taken into account when determining machine assignments. Such preferences can include optimizing various performance categories (e.g., fuel efficiency, costs such as operation cost, soil compaction, etc.), using a given machine, or given machine type/model, for certain quality cotton, etc. Thus, in one example, machine assignment logic 650 can utilize user or operator inputs 644 in identifying machine assignments, as will be discussed in further detail below.

As shown in FIG. 6, logistics system 510 can obtain or utilize various other data 648, including, but not limited to, other data described herein. In one example, other data can include maps of the worksite, the maps of the worksite can indicate or include values of various characteristics (e.g., crop characteristics, terrain characteristics, etc.). The maps may be generated prior to the cotton harvesting operation, such as based on overhead imagery, based on sensor data collected during prior operations, user/operator input, as well as generated in various other ways.

As illustrated in FIG. 6, logistics system 510 includes machine assignment logic 650, route generator 652, map generator 654, and can include other items and functionality 658 as well.

Machine assignment logic 650 illustratively determines assignments of cotton collection & transport machines 400. Assignments can include, assigning one or more cotton collection & transport machines 400 to select modules or other cotton clusters or select worksites, or both. Assignments can include assigning one or more cotton collection & transport machines to cotton groupings. In determining machine assignments, machine assignment logic 650 can take into account one or more of cotton quality outputs 630, cotton collection & transport machine sensor data 640, machine data 642, user or operator inputs 644, as well as various other data 648. A respective set of one or more cotton collection & transport machines 400 may be assigned to each distinctive cotton grouping identified by cotton quality classifier system 310 and indicated by outputs 630. Thus, the modules or other cotton clusters corresponding to a given cotton grouping will be collected and transported by the corresponding (assigned) set of one or more cotton collection & transport machines 400.

Machine assignment logic 650 may assign cotton collection & transport machines 400 based on sensor data 640. For instance, machine assignment logic 650 may assign cotton collection & transport machines 400 based on their current geographic locations (e.g., select the one or more cotton collection & transport machines 400 that are closest in location to the location(s) of a given set of cotton modules or other cotton clusters (e.g., cotton grouping) as indicated by outputs 630). Machine assignment logic 650 may assign cotton collection & transport machines 400 based on machine data 642. For instance, machine assignment logic 650 may assign cotton collection & transport machines 400 based on their carrying capacities, performance capabilities, machine dimensions, etc. For example, machine assignment logic 650 may assign cotton collection & transport machines 400 to a given set of modules or other cotton clusters (e.g., cotton grouping) based on the number or quantity of modules or other cotton clusters (as indicated by outputs 630) and the machine data 642 such that the given set of modules or other cotton clusters can be collected and transported efficiently. Machine assignment logic 650 may assign cotton collection & transport machines 400 based on user or operator inputs 644. For instance, machine assignment logic 650 may assign cotton collection & transport machines 400 based on user or operator preferences, such as performance optimization preferences (e.g., fuel efficiency, cost, time to complete, etc.) or preferences for use of particular models/types for the cotton quality corresponding to a given set of cotton modules or other cotton clusters (e.g., cotton grouping). These are merely some examples.

Each machine assignment output by machine assignment logic 650 can include the identification of the one or more cotton collection & transport machines 400 being assigned and the worksite(s), cotton module(s) or other cotton cluster(s), or cotton groupings to which they are being assigned for collection and transport.

Route generator 652 illustratively generates routes for the assigned cotton collection & transport machines 400 to the worksites, cotton module(s) or other cotton cluster(s), or cotton groupings to which they are assigned. The routes can be used in the control of the cotton collection & transport machines 400 or can provided for presentation to operators 360 or users 366, or both. In some examples, route generator 652 may account for current locations (as indicated by sensor data 640), machine performance capabilities (e.g., machine ratings) (as indicated by machine data 642), user or operator inputs 644 (e.g., performance optimization preferences such as fuel efficiency, cost, time to complete, etc.) when generating the routes. For example, route generator 652 may generate routes the quickest (e.g., shortest distance, least total time, least idle time, etc.) routes, the routes that limit machine wear, the most fuel-efficient routes, as well as various other routes.

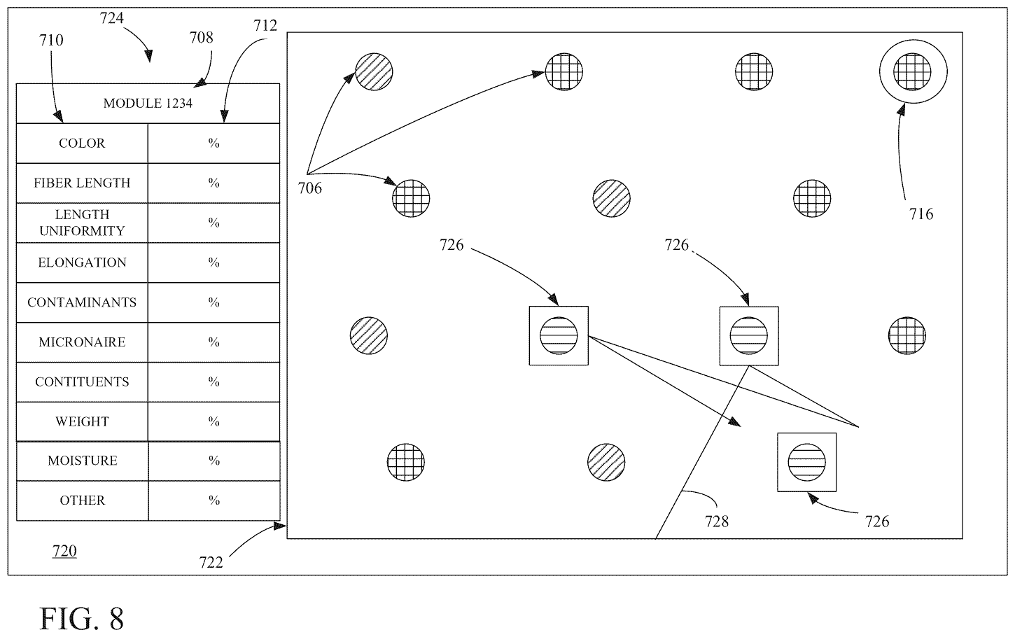

Map generator 654 illustratively generates cotton quality logistics maps, such as a cotton quality logistics map of worksite (e.g., field), that includes indicators (e.g., icons, etc.) showing the location of cotton modules or other cotton clusters at the worksite, indicates the cotton quality metric values or grouping, or both, for each module or other cotton cluster, and includes indicators for machine assignments or routes, or both. Thus, in one example, the maps generated by map generator 654 can be similar to maps generated by map generator 610 but can further include indicators for machine assignments or routes, or both. In one example, map generator 654 may obtain a map generated by map generator 610 and modify it (e.g., add indicators for machine assignments or routes, or both) to generate a map. The maps generated by map generator 654 may be provided to other items of system 300 and/or may be presented to an operator or user, or both, via interface mechanisms. One example of a map generated by map generator 654 is shown in FIG. 8.

As can be seen, logistics system 510 is operable to generate one or more cotton quality logistics outputs 660. The one or more cotton quality logistics outputs 660 can include one or more machine assignments, one or more routes, or one or more cotton quality logistics maps, or any combination thereof. The one or more cotton quality logistics outputs 660 can be provided to other items of system 300 and can be used in the control of one or more mobile machines (e.g., 100 or 400, or both), The one or more cotton quality logistics outputs 660 can be presented to an operator or user (e.g., 360 or 366), or both, via a corresponding interface mechanism (e.g., 305/405 or 364, or a combination thereof).

FIG. 7 is a pictorial illustration showing one example interface display 700 including a map display portion 702 and a cotton quality characteristics display portion 704. It will be understood that display 700 can be displayed on interface mechanisms, such as operator interface mechanisms 305, operator interface mechanisms 405, or user interface mechanisms 364, or a combination thereof. In one example, display 700 comprises a graphical user interface display.

Map display portion 702 illustratively includes a plurality of cotton cluster indicators 706, each indicator 706 displayed at a location on the map display portion corresponding to the location, in the worksite, of the corresponding cotton cluster (e.g., module, etc.) that the indicator 706 represents. Additionally, as can be seen, each indicator 706 can have a given visual characteristic (illustratively shown as a given pattern). The visual characteristic can indicate the cotton grouping to which a given indicator 706 (and thus a given cotton cluster (e.g., module, etc.)) corresponds to. While patterns are shown, it will be understood that in other examples, various other visual characteristics can be utilized, such as coloring, shading, etc. The map shown in FIG. 7 is one example of a cotton quality map generated by map generator 610.

Cotton quality characteristics display portion 704 is illustratively shown as a table that includes a cluster identification portion 708 that indicates the ID of the cotton cluster (e.g., module, etc.) to which the table corresponds, a cotton quality characteristics column 710 having a plurality of rows, each row listing a given cotton quality characteristic, and a cotton quality metrics column 712 having a plurality of rows, each row listing a cotton quality metric corresponding to a row (or cotton quality characteristic) of the cotton quality characteristics column 710. While the examples show the cotton quality metrics as being percentages, in other examples, other types of metrics could be used, further, in some examples, some metrics can be different in type than other metrics. While a table is shown in FIG. 7, this is for example only. In other examples, cotton quality characteristics display portion 704 can be in other forms, such as graphs, charts, or other types of data display elements. Additionally, while cotton quality characteristics display portion is shown separated from map display portion 702 in the illustrated examples, in other examples, cotton quality characteristic display portion 704 could be displayed in map display portion 702, such as adjacent to or over the corresponding cotton cluster indicator.

Additionally, the given cotton cluster (e.g., module, etc.) to which portion 704 corresponds can be visually indicated by an indicator 716. While indicator 716 is illustratively a bounding indicator, this is merely one example. In other examples, indicator 716 could be an overlay, flashing or blinking of the given cluster indicator 706, a change in a visual characteristic of the given cluster indicator 706 (e.g., change color, boldening, changing transparency, etc.), a change in a visual characteristic of the other cluster indicators 706, as well as various other types of indicators.

FIG. 8 is a pictorial illustration showing one example interface display 720 including a map display portion 722 and a cotton quality characteristics display portion 724. It will be understood that display 720 can be displayed on interface mechanisms, such as operator interface mechanisms 305, operator interface mechanisms 405, or user interface mechanisms 364, or a combination thereof. In one example, display 720 comprises a graphical user interface display.

Cotton quality characteristics display portion 724 is similar to cotton quality characteristics display portion 704 and therefore similar items are numbered similarly. Map display portion 722 is similar to map display portion 702 and thus similar items are numbered similarly. It can be seen in FIG. 8 that map display portion 722 further includes (in addition to cluster indicators 706 and indicator 716) machine assignment indicators 726 and a route indicator 728. The map shown in FIG. 8 is an example of a cotton quality logistics map generated by map generator 654. It will be understood that in some examples, a cotton quality logistics map need not include some of the items shown in the example of FIG. 8. For instance, a cotton quality logistics map need not include indicator 716, other cotton cluster indicators 706 to which the particular machine 400 is not assigned, nor potion 724.

Machine assignment indicators 726 illustratively visually indicate the cotton cluster indicators 706 (and thus the cotton clusters (e.g., modules, etc.) or cotton grouping) to which a given set of one or more cotton collection & transport machines 400 are assigned to. While indicators 726 are illustratively bounding indicators, this is merely one example. In other examples, indicators 726 could be overlays, flashing or blinking of the given cluster indicators 706, a change in a visual characteristic of the given cluster indicators 706 (e.g., change color, boldening, changing transparency, etc.), a change in a visual characteristic of the other cluster indicators 706, as well as various other types of indicators.

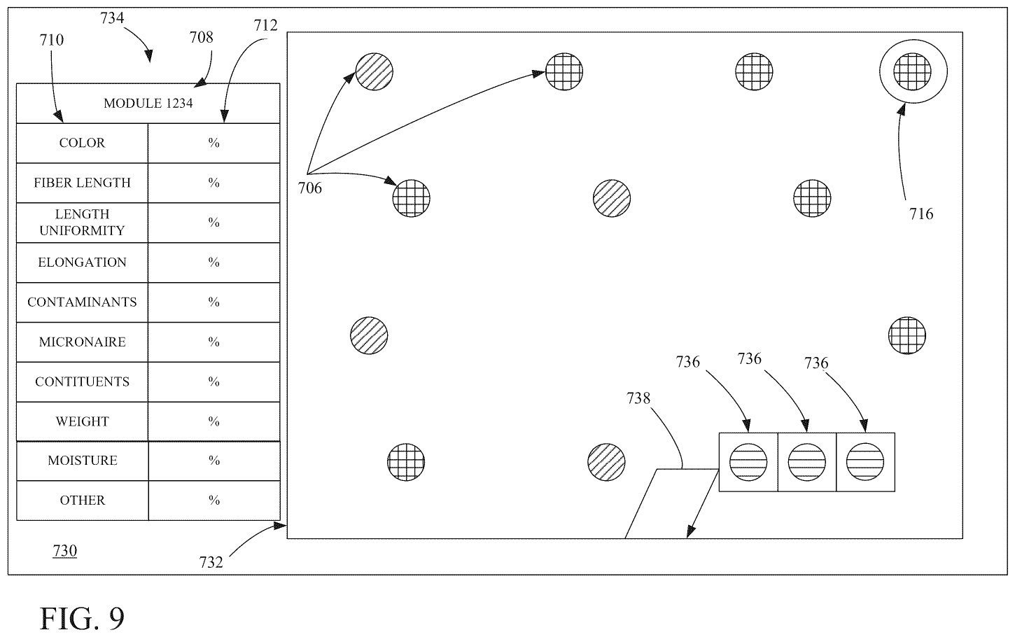

Route indictor 728 illustratively visually indicates a route that a cotton collection & transport machine 400 is to travel at the worksite to which the map corresponds to collect and transport the cotton clusters (or cotton grouping) to which the machine 400 is assigned. In the illustrated example, the route indicator 728 corresponds to a route that the machine 400 is to travel to collect and transport the cotton modules to another location on the worksite for pickup by another machine 400, as will be shown in FIG. 9.

FIG. 9 is a pictorial illustration showing one example interface display 730 including a map display portion 732 and a cotton quality characteristics display portion 734. It will be understood that display 730 can be displayed on interface mechanisms, such as operator interface mechanisms 305, operator interface mechanisms 405, or user interface mechanisms 364, or a combination thereof. In one example, display 730 comprises a graphical user interface display.

Cotton quality characteristics display portion 734 is similar to cotton quality characteristics display portion 704 and thus similar items are numbered similarly. Map display portion 732 is similar to map display portion 702 and thus similar items are numbered similarly. It can be seen in FIG. 9 that map display portion 732 further includes (in addition to cluster indicators 706 and indicator 716) machine assignment indicators 736 and a route indicator 738. The map shown in FIG. 9 is an example of a cotton quality logistics map generated by map generator 654.

It will be understood that in some examples, a cotton quality logistics map need not include some of the items shown in the example of FIG. 9. For instance, a cotton quality logistics map need not include indicator 716, other cotton cluster indicators 706 to which the particular machine 400 is not assigned, nor potion 734.

Machine assignment indicators 736 illustratively visually indicate the cotton cluster indicators 706 (and thus the cotton clusters (e.g., modules, etc.) or cotton grouping) to which a given set of one or more cotton collection & transport machines 400 are assigned to. While indicators 736 are illustratively bounding indicators, this is merely one example. In other examples, indicators 736 could be overlays, flashing or blinking of the given cluster indicators 706, a change in a visual characteristic of the given cluster indicators 706 (e.g., change color, boldening, changing transparency, etc.), a change in a visual characteristic of the other cluster indicators 706, as well as various other types of indicators.

Route indictor 738 illustratively visually indicates a route that a cotton collection & transport machine 400 is to travel at the worksite to which the map corresponds to collect and transport the cotton clusters (or cotton grouping) to which the machine 400 is assigned. In the illustrated example, the route indicator 738 corresponds to a route that the machine 400 is to travel to collect cotton modules, previously moved closer together (as described in FIG. 8), and transport them away from the worksite.

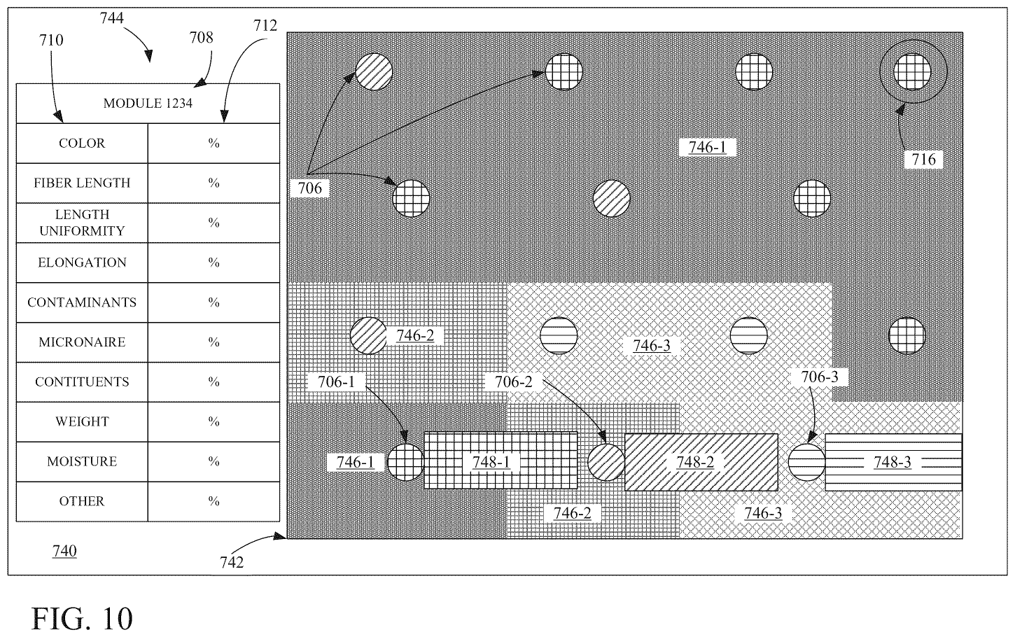

FIG. 10 is a pictorial illustration showing one example interface display 740 including a map display portion 742 and a cotton quality characteristics display portion 744. It will be understood that display 740 can be displayed on interface mechanisms, such as operator interface mechanisms 305, operator interface mechanisms 405, or user interface mechanisms 364, or a combination thereof. In one example, display 740 comprises a graphical user interface display.

Cotton quality characteristics display portion 744 is similar to cotton quality characteristics display portion 704 and thus similar items are numbered similarly. Map display portion 742 is similar to map display portion 702 and thus similar items are numbered similarly. It can be seen in FIG. 10 that map display portion 742 further includes (in addition to cluster indicators 706 and indicator 716) characteristic value indicators 746 (illustratively 746-1, 746-2, and 746-3) and harvest area indicators 748 (illustratively 748-1, 748-2, and 748-3). The map shown in FIG. 10 is one example of a cotton quality map generated by map generator 610.

Characteristic value indicators 746 illustratively visually indicate values of a characteristic relative to the worksite, such as a crop characteristic (e.g., yield, crop genotype (e.g., hybrid, cultivar, etc.), crop moisture, etc.), a terrain characteristic (e.g., topography, soil characteristic, etc.), as well as various other characteristics. The characteristic values can be derived from a map of the worksite, such as one of the maps described as part of other data 605 or other data 648, or another type of map. The characteristic values can be derived in various other ways, such as based on sensor data generated during prior operations at the worksite, or based on operator or user input. In the illustrated example, characteristic value indicators 746 indicate yield values. 746-1 illustratively represents high yield, 746-2 illustratively represents medium yield, and 746-3 illustratively represents low yield. High, medium, and low are merely examples of values. It can be seen that each indicator 746 can have a given visual characteristic (illustratively shown as a pattern). The visual characteristic can correspond to the value.

Harvest area indicators 748 illustratively visually indicate the area of the worksite from which cotton was collected to generate a corresponding cotton cluster. In the illustrated example, harvest area indicator 748-1 indicates the area of the worksite from which cotton was collected to generate the cotton cluster represented by indicator 706-1. In the illustrated example, harvest area indicator 748-2 indicates the area of the worksite from which cotton was collected to generate the cotton cluster represented by indicator 706-2. In the illustrated example, harvest area indicator 748-3 indicates the area of the worksite from which cotton was collected to generate the cotton cluster represented by indicator 706-3. It can be seen that each indicator 748 can have a given visual characteristic (illustratively shown as a pattern). In the illustrated example, the visual characteristic of each indicator 748 matches the visual characteristic of the corresponding indicator 706, though this not need be the case. Additionally, while the illustrated example only shows a limited number of indicators 748 for the sake of illustrative clarity, an indicator 748 can be provided for each cotton cluster (e.g., an indicator 738 for each indicator 706).

Though not shown, it will be understood that a map display of a cotton quality logistics map, such as the example shown in FIGS. 8-9, can also include characteristic value indicator 746 and harvest area indicators 748.

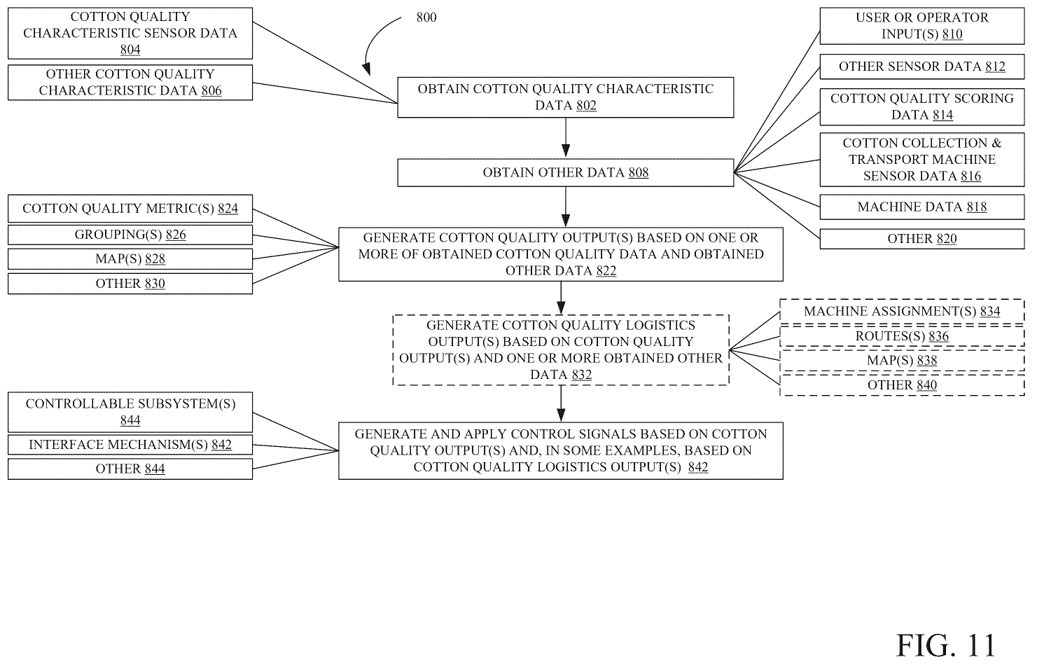

FIG. 11 shows a flowchart illustrating one example operation 800 of system 300.

At block 802, one or more items of cotton quality characteristic data are obtained (e.g., retrieved or received) by system 300 (e.g., cotton quality classifier system 310). As indicated by block 804, cotton quality characteristic sensor data 601 can be obtained. As indicated by block 806, other cotton quality characteristic data 602 can be obtained.

At block 808, one or more items of other data are obtained (e.g., retrieved or received) by system 300 (e.g., cotton quality classifier system 310 or logistics system 510, or both). As indicated by block 810, user or operator inputs 600 or 644, or both, can be obtained. As indicated by block 812, other sensor data 603 can be obtained. As indicated by block 814, cotton quality scoring data 604 can be obtained. As indicated by block 816, cotton collection & transport machine sensor data 640 can be obtained. As indicated by block 818, machine data 642 can be obtained. As indicated by block 820, various other data (e.g., 605 or 648, or both, etc.) can be obtained.

At block 822, system 300 (e.g., cotton quality classifier system 310) generates one or more cotton quality outputs 630 based on one or more items of the cotton quality characteristic data obtained at block 802 and, in some examples, based on one or more items of other data obtained at block 808 (e.g. user or operator inputs 600, other sensor data 603, cotton quality scoring data 604, and other data 605). As indicated by block 824, the cotton quality outputs 630 can include one or more cotton quality metrics. As indicated by block 826, the cotton quality outputs 630 can, additionally or alternatively, include one or more cotton groupings. As indicated by block 828, the cotton quality outputs 630 can, alternatively or additionally, include one or more cotton quality maps (e.g., such as the example shown in FIG. 7, the example shown in FIG. 10, etc.). As indicated by block 830, the cotton quality outputs 630 can include various other items.

In some examples, method 800 includes block 832. At block 832, system 300 (e.g., logistics system 510) generates one or more logistics outputs 660 based on the one or more cotton quality outputs 630 and, in some examples, based on one or more items of other data obtained at block 808 (e.g., user or operator inputs 644, cotton collection & transport machine sensor data 640, machine data 642, and other data 648). As indicated by block 834, the cotton quality logistics outputs 660 can include one or more machine assignments. As indicated by block 836, the cotton quality logistics outputs 630 can, additionally or alternatively, include one or more routes As indicated by block 838, the cotton quality logistics outputs 630 can, alternatively or additionally, include one or more cotton quality logistics maps (e.g., such as the example shown in FIG. 8, the example shown in FIG. 9, etc.). As indicated by block 840, the cotton quality logistics outputs 640 can include various other items.

At block 842, system 300 (e.g., control system 314 or control system 414, or both) generate an apply one or more control signals based on the one or more cotton quality outputs 630 and, in some examples, based on the one or more cotton quality logistics outputs 660. As indicated by block 844, the one or more control signals can be generated to control one or more controllable subsystems, such as controllable subsystems 316 (e.g., tagger 350) or one or more controllable subsystems 416, or both. As indicated by block 846, the one or more control signals can, alternatively or additionally, be generated to control one or more interface mechanisms, such as one or more interface mechanisms 305 or one or more interface mechanisms 364, or both, to present cotton quality outputs 630 (or information thereof) or cotton quality logistics outputs 660, or both. The presentations can include presenting one or more of cotton quality metrics, cotton groupings, machine assignments, routes, maps (e.g., cotton quality maps or cotton quality logistics maps, or both). The presentations can be in the form of displays, such as a display 700, a display 720, a display 730, or a display 740, or another display. As indicated by block 844, the one or more control signals can, alternatively or additionally, be generated to control various other items of system 300, for example, but not by limitation, one or more of communication system 306, communication system 406, and communication system 506 to communicate information with other items of system 300 (e.g., communicated over networks 359).

The present discussion has mentioned processors and servers. In some examples, the processors and servers include computer processors with associated memory and timing circuitry, not separately shown. They are functional parts of the systems or devices to which they belong and are activated by and facilitate the functionality of the other components or items in those systems.

Also, a number of user interface displays have been discussed. The displays can take a wide variety of different forms and can have a wide variety of different user actuatable operator interface mechanisms disposed thereon. For instance, user actuatable operator interface mechanisms may include text boxes, check boxes, icons, links, drop-down menus, search boxes, etc. The user actuatable operator interface mechanisms can also be actuated in a wide variety of different ways. For instance, they can be actuated using operator interface mechanisms such as a point and click device, such as a track ball or mouse, hardware buttons, switches, a joystick or keyboard, thumb switches or thumb pads, etc., a virtual keyboard or other virtual actuators. In addition, where the screen on which the user actuatable operator interface mechanisms are displayed is a touch sensitive screen, the user actuatable operator interface mechanisms can be actuated using touch gestures. Also, user actuatable operator interface mechanisms can be actuated using speech commands using speech recognition functionality. Speech recognition may be implemented using a speech detection device, such as a microphone, and software that functions to recognize detected speech and execute commands based on the received speech.

A number of data stores have also been discussed. It will be noted the data stores can each be broken into multiple data stores. In some examples, one or more of the data stores May be local to the systems accessing the data stores, one or more of the data stores may all be located remote form a system utilizing the data store, or one or more data stores may be local while others are remote. All of these configurations are contemplated by the present disclosure.

Also, the figures show a number of blocks with functionality ascribed to each block. It will be noted that fewer blocks can be used to illustrate that the functionality ascribed to multiple different blocks is performed by fewer components. Also, more blocks can be used illustrating that the functionality may be distributed among more components. In different examples, some functionality may be added, and some may be removed.

It will be noted that the above discussion has described a variety of different systems, generators, models, logic, controllers, components, and interactions. It will be appreciated that any or all of such systems, generators, models, logic, controllers, components, and interactions may be implemented by hardware items, such as one or more processors, one or more processors executing computer executable instructions stored in memory, memory, or other processing components, some of which are described below, that perform the functions associated with those systems, generators, models, logic, controllers, components, or interactions. In addition, any or all of the systems, generators, models, logic, controllers, components, and interactions may be implemented by software that is loaded into a memory and is subsequently executed by one or more processors or one or more servers or other computing component(s), as described below. Any or all of the systems, generators, models, logic, controllers, components, and interactions may also be implemented by different combinations of hardware, software, firmware, etc., some examples of which are described below. These are some examples of different structures that May be used to implement any or all of the systems, generators, models, logic, controllers, components, and interactions described above. Other structures may be used as well.

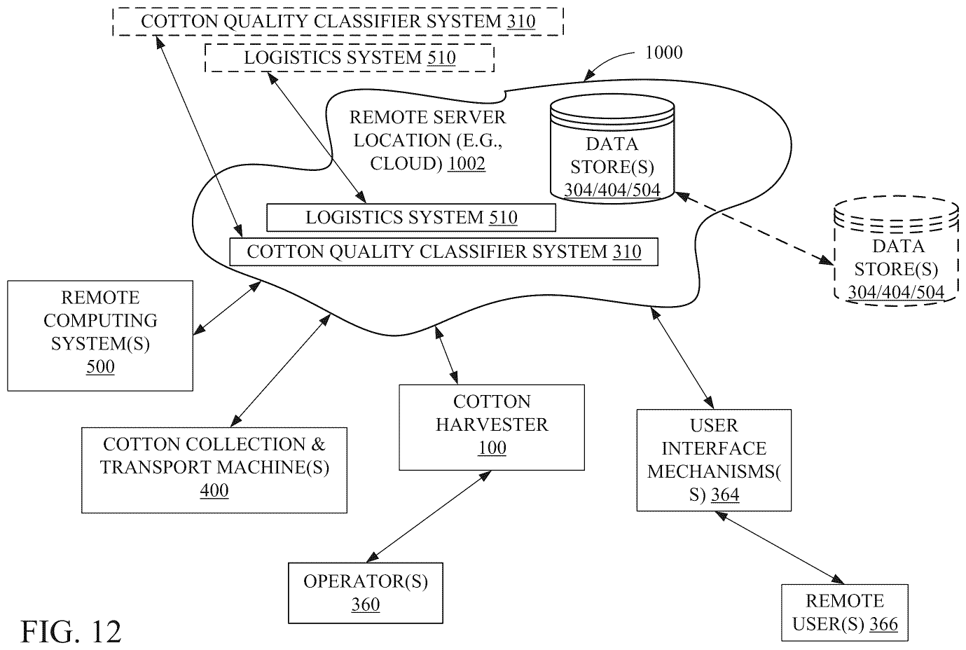

FIG. 12 is a block diagram of a remote server architecture 1000. FIG. 12, also shows cotton harvester 100, one or more cotton collection & transport machines 400, one or more remote computing systems 500, and one or more remote user interface mechanisms 364 in communication with the remote server environment. The cotton harvester 100, one or more cotton collection & transport machines 400, one or more remote computing systems 500, and one or more remote user interface mechanisms 364 communicate with elements in a remote server architecture 1000. In some examples, remote server architecture 1000 provides computation, software, data access, and storage services that do not require end-user knowledge of the physical location or configuration of the system that delivers the services. In various examples, remote servers may deliver the services over a wide area network, such as the internet, using appropriate protocols. For instance, remote servers may deliver applications over a wide area network and may be accessible through a web browser or any other computing component. Software or components shown in previous figures as well as data associated therewith, may be stored on servers at a remote location. The computing resources in a remote server environment may be consolidated at a remote data center location, or the computing resources may be dispersed to a plurality of remote data centers. Remote server infrastructures may deliver services through shared data centers, even though the services appear as a single point of access for the user. Thus, the components and functions described herein may be provided from a remote server at a remote location using a remote server architecture. Alternatively, the components and functions may be provided from a server, or the components and functions can be installed on client devices directly, or in other ways.

In the example shown in FIG. 12, some items are similar to those shown in previous figures and those items are similarly numbered. FIG. 12 specifically shows that cotton quality classifier system 310, logistics system 510, and one or more of data stores 304, data stores 304, and data stores 504 may be located at a server location 1002 that is remote from the mobile work cotton harvester 100, one or more cotton collection & transport machines 400, one or more remote computing systems 500, and one or more remote user interface mechanisms 364. Therefore, in the example shown in FIG. 12, cotton harvester 100, one or more cotton collection & transport machines 400, one or more remote computing systems 500, and one or more remote user interface mechanisms 364 access systems through remote server location 1002. In other examples, various other items may also be located at server location 1002, such as various other items of system 300.

FIG. 12 also depicts another example of a remote server architecture. FIG. 12 shows that some elements of previous figures may be disposed at a remote server location 1002 (e.g., cloud) 1002 while others may be located elsewhere. By way of example, one or more of data stores 304, 404, and 504 may be disposed at a location separate from location 1002 and accessed via the remote server at location 1002. Similarly, one or more of cotton quality classifier system 310 and logistics system 510 may be disposed at a location separate from locations 1002 and accessed via the remote server at locations 1002. Regardless of where the elements are located, the elements can be accessed directly by cotton harvester 100, one or more cotton collection & transport machines 400, one or more remote computing systems 500, and one or more remote user interface mechanisms 364 through a network such as a wide area network or a local area network; the elements can be hosted at a remote site by a service; or the elements can be provided as a service or accessed by a connection service that resides in a remote location. Also, data may be stored in any location, and the stored data may be accessed by, or forwarded to, operators, users, or systems. For instance, physical carriers may be used instead of, or in addition to, electromagnetic wave carriers. In some examples, where wireless telecommunication service coverage is poor or nonexistent, another machine, such as a fuel truck or other mobile machine or vehicle, may have an automated, semi-automated or manual information collection system. As a mobile machine (e.g., cotton harvester 100, cotton collection & transport machine 400) comes close to the machine containing the information collection system, such as a fuel truck prior to fueling, the information collection system collects the information from the mobile machine using any type of ad-hoc wireless connection. The collected information may then be forwarded to another network when the machine containing the received information reaches a location where wireless telecommunication service coverage or other wireless coverage—is available. For instance, a fuel truck may enter an area having wireless communication coverage when traveling to a location to fuel other machines or when at a main fuel storage location. All of these architectures are contemplated herein. Further, the information may be stored on a mobile machine until the mobile machine enters an area having wireless communication coverage. The mobile machine, itself, May send the information to another network.

It will also be noted that the elements of previous figures, or portions thereof, May be disposed on a wide variety of different devices. One or more of those devices may include an on-board computer, an electronic control unit, a quantum computer, a display unit, a server, a desktop computer, a laptop computer, a tablet computer, or other mobile device, such as a palm top computer, a cell phone, a smart phone, a multimedia player, a personal digital assistant, etc.

In some examples, remote server architecture 1000 may include cybersecurity measures. Without limitation, these measures may include encryption of data on storage devices, encryption of data sent between network nodes, authentication of people or processes accessing data, as well as the use of ledgers for recording metadata, data, data transfers, data accesses, and data transformations. In some examples, the ledgers may be distributed and immutable (e.g., implemented as blockchain).

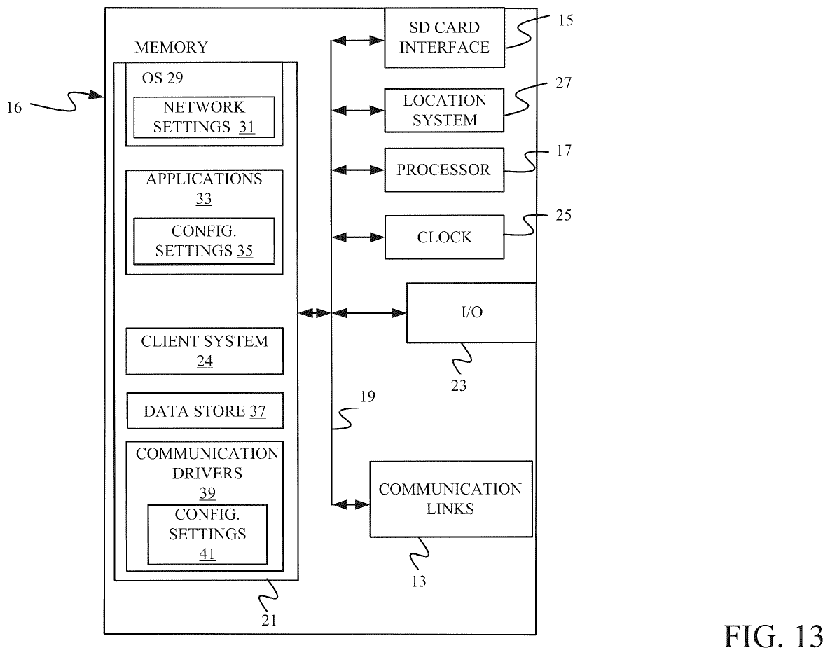

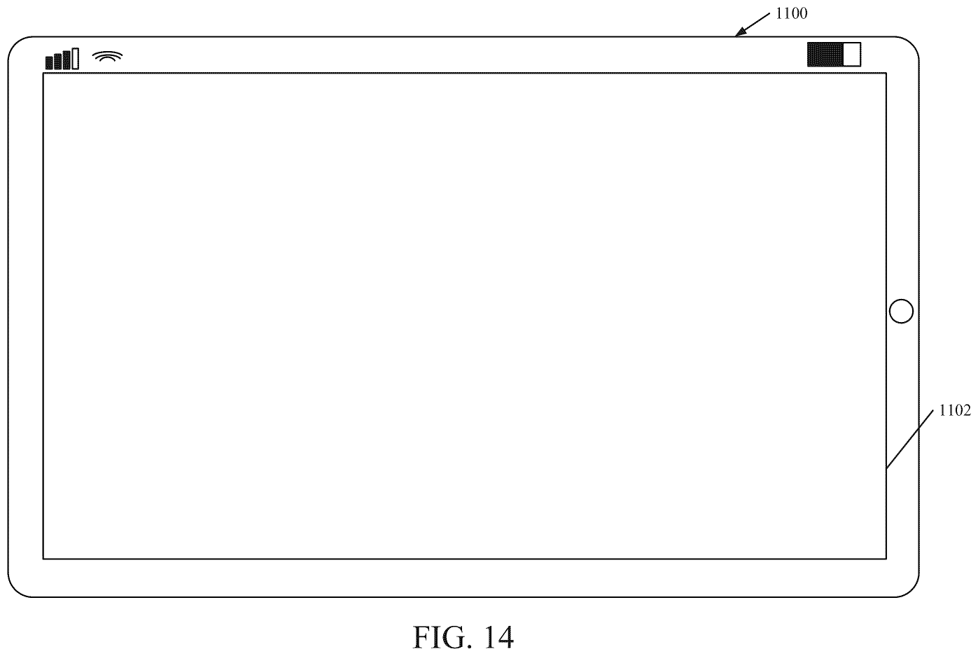

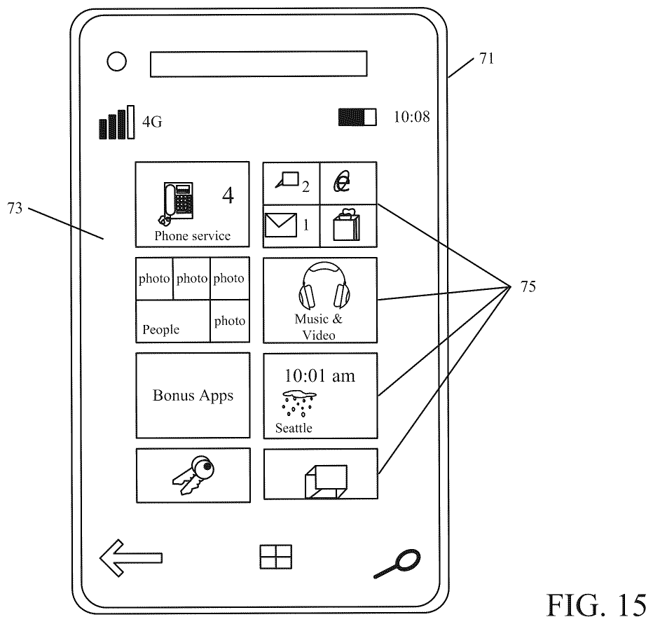

FIG. 13 is a simplified block diagram of one illustrative example of a handheld or mobile computing device that can be used as a user's or client's handheld device 16, in which the present system (or parts of it) can be deployed. For instance, a mobile device can be deployed in the operator compartment of a mobile machine (e.g., a cotton harvester 100, cotton collection & transport machine 400) for use in generating, processing, or displaying the outputs 630 or outputs 660, or both, discussed above. FIGS. 14-15 are examples of handheld or mobile devices.

FIG. 13 provides a general block diagram of the components of a client device 16 that can run some components shown in previous figures, that interact with them, or both. In the device 16, a communications link 13 is provided that allows the handheld device to communicate with other computing devices and under some examples provides a channel for receiving information automatically, such as by scanning. Examples of communications link 13 include allowing communication though one or more communication protocols, such as wireless services used to provide cellular access to a network, as well as protocols that provide local wireless connections to networks.

In other examples, applications can be received on a removable Secure Digital (SD) card that is connected to an interface 15. Interface 15 and communication links 13 communicate with a processor 17 (which can also embody processors or servers from other figures) along a bus 19 that is also connected to memory 21 and input/output (I/O) components 23, as well as clock 25 and location system 27.

I/O components 23, in one example, are provided to facilitate input and output operations. I/O components 23 for various examples of the device 16 can include input components such as buttons, touch sensors, optical sensors, microphones, touch screens, proximity sensors, accelerometers, orientation sensors and output components such as a display device, a speaker, and or a printer port. Other I/O components 23 can be used as well.

Clock 25 illustratively comprises a real time clock component that outputs a time and date. It can also, illustratively, provide timing functions for processor 17.

Location system 27 illustratively includes a component that outputs a current geographical location of device 16. This can include, for instance, a global positioning system (GPS) receiver, a LORAN system, a dead reckoning system, a cellular triangulation system, or other positioning system. Location system 27 can also include, for example, mapping software or navigation software that generates desired maps, navigation routes and other geographic functions.

Memory 21 stores operating system 29, network settings 31, applications 33, application configuration settings 35, data store 37, client system 24, communication drivers 39, and communication configuration settings 41. Memory 21 can include all types of tangible volatile and non-volatile computer-readable memory devices. Memory 21 may also include computer storage media (described below). Memory 21 stores computer readable instructions that, when executed by processor 17, cause the processor to perform computer-implemented steps or functions according to the instructions. Processor 17 may be activated by other components to facilitate their functionality as well. 2

FIG. 14 shows one example in which device 16 is a tablet computer 1100. In FIG. 14, computer 1100 is shown with user interface display screen 1102. Screen 1102 can be a touch screen or a pen-enabled interface that receives inputs from a pen or stylus. Tablet computer 1100 may also use an on-screen virtual keyboard. Of course, computer 1100 might also be attached to a keyboard or other user input device through a suitable attachment mechanism, such as a wireless link or USB port, for instance. Computer 1100 may also illustratively receive voice inputs as well.

FIG. 15 is similar to FIG. 14 except that the device is a smart phone 71. Smart phone 71 has a touch sensitive display 73 that displays icons or tiles or other user input mechanisms 75. Mechanisms 75 can be used by a user to run applications, make calls, perform data transfer operations, etc. In general, smart phone 71 is built on a mobile operating system and offers more advanced computing capability and connectivity than a feature phone.

Note that other forms of the devices 16 are possible.

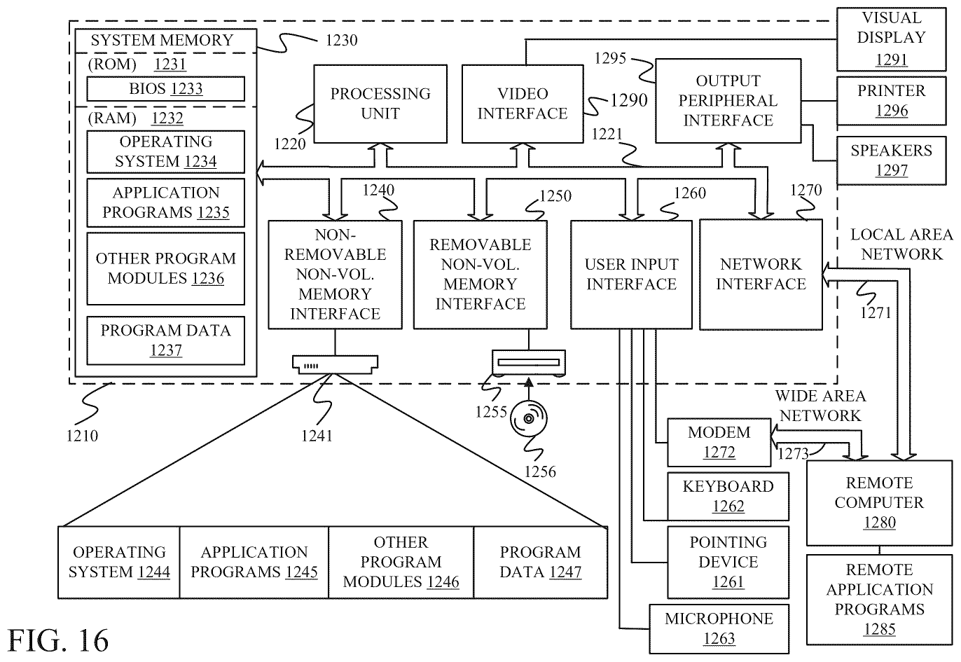

FIG. 16 is one example of a computing environment in which elements of previous figures described herein can be deployed. With reference to FIG. 16, an example system for implementing some embodiments includes a computing device in the form of a computer 1210 programmed to operate as discussed above. Components of computer 1210 may include, but are not limited to, a processing unit 1220 (which can comprise processors or servers from previous figures), a system memory 1230, and a system bus 1221 that couples various system components including the system memory to the processing unit 1220. The system bus 1221 may be any of several types of bus structures including a memory bus or memory controller, a peripheral bus, and a local bus using any of a variety of bus architectures. Memory and programs described with respect to previous figures described herein can be deployed in corresponding portions of FIG. 16.

Computer 1210 typically includes a variety of computer readable media. Computer readable media may be any available media that can be accessed by computer 1210 and includes both volatile and nonvolatile media, removable and non-removable media. By way of example, and not limitation, computer readable media may comprise computer storage media and communication media. Computer storage media is different from, and does not include, a modulated data signal or carrier wave. Computer readable media includes hardware storage media including both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 1210. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 1230 includes computer storage media in the form of volatile and/or nonvolatile memory or both such as read only memory (ROM) 1231 and random access memory (RAM) 1232. A basic input/output system 1233 (BIOS), containing the basic routines that help to transfer information between elements within computer 1210, such as during start-up, is typically stored in ROM 1231. RAM 1232 typically contains data or program modules or both that are immediately accessible to and/or presently being operated on by processing unit 1220. By way of example, and not limitation, FIG. 16 illustrates operating system 1234, application programs 1235, other program modules 1236, and program data 1237.

The computer 1210 may also include other removable/non-removable volatile/nonvolatile computer storage media. By way of example only, FIG. 16 illustrates a hard disk drive 1241 that reads from or writes to non-removable, nonvolatile magnetic media, an optical disk drive 1255, and nonvolatile optical disk 1256. The hard disk drive 1241 is typically connected to the system bus 1221 through a non-removable memory interface such as interface 1240, and optical disk drive 1255 are typically connected to the system bus 1221 by a removable memory interface, such as interface 1250.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 16, provide storage of computer readable instructions, data structures, program modules and other data for the computer 1210. In FIG. 16, for example, hard disk drive 1241 is illustrated as storing operating system 1244, application programs 1245, other program modules 1246, and program data 1247. Note that these components can either be the same as or different from operating system 1234, application programs 1235, other program modules 1236, and program data 1237.

A user may enter commands and information into the computer 1210 through input devices such as a keyboard 1262, a microphone 1263, and a pointing device 1261, such as a mouse, trackball or touch pad. Other input devices (not shown) may include a joystick, game pad, satellite dish, scanner, or the like. These and other input devices are often connected to the processing unit 1220 through a user input interface 1260 that is coupled to the system bus, but may be connected by other interface and bus structures. A visual display 1291 or other type of display device is also connected to the system bus 1221 via an interface, such as a video interface 1290. In addition to the monitor, computers may also include other peripheral output devices such as speakers 1297 and printer 1296, which may be connected through an output peripheral interface 1295.

The computer 1210 is operated in a networked environment using logical connections (such as a controller area network—CAN, local area network—LAN, or wide area network WAN) to one or more remote computers, such as a remote computer 1280.

When used in a LAN networking environment, the computer 1210 is connected to the LAN 1271 through a network interface or adapter 1270. When used in a WAN networking environment, the computer 1210 typically includes a modem 1272 or other means for establishing communications over the WAN 1273, such as the Internet. In a networked environment, program modules may be stored in a remote memory storage device. FIG. 16 illustrates, for example, that remote application programs 1285 can reside on remote computer 1280.

It should also be noted that the different examples described herein can be combined in different ways. That is, parts of one or more examples can be combined with parts of one or more other examples. All of this is contemplated herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of the claims.

What is claimed is:

1. A cotton harvesting system comprising:
one or more cotton quality characteristic sensors, on-board a cotton harvester, configured to detect a plurality of cotton quality characteristics and to generate cotton quality characteristic sensor data indicative of the detected plurality of cotton quality characteristics;
one or more processors; and
memory storing computer executable instructions, executable by the one or more processors, the computer executable instructions, when executed by the one or more processors, causing the one or more processors perform steps comprising:
obtaining the cotton quality characteristic sensor data;
determining, for each cotton cluster of a plurality of cotton clusters generated by the cotton harvester, a respective set of cotton quality metrics based on the cotton quality characteristic sensor data, wherein, for each respective set, each cotton quality metric corresponds to a different cotton quality characteristic of the plurality of cotton quality characteristics than the other cotton quality metrics of the respective set; and
generating a control signal based on at least one respective set of cotton quality metrics.

2. The cotton harvesting system of claim 1, wherein the one or more cotton quality characteristic sensors comprise one or more of:
near infrared (NIR) sensors;
terahertz sensors;
light sensors; or
a high volume instrument sensor system.

3. The cotton harvesting system of claim 1, wherein the plurality of cotton quality characteristics comprises two or more of:
color;
fiber length;
fiber length uniformity;
elongation;
contaminants;
micronaire; or
constituents.

4. The cotton harvesting system of claim 1, wherein the computer executable instructions, when executed by the one or more processors, further cause the one or more processors to perform a step comprising:
providing the respective set of cotton quality metrics for each cotton cluster of the plurality of cotton clusters generated by the cotton harvester to a cotton grouping model to obtain, as a cotton grouping model output, a plurality of cotton groupings, each cotton grouping, of the plurality of cotton groupings, comprising a different set of cotton clusters of the plurality of cotton clusters generated by the cotton harvester.

5. The cotton harvesting system of claim 4, wherein the cotton grouping model utilizes a K-means clustering algorithm.

6. The cotton harvesting system of claim 4, wherein the computer executable instructions, when executed by the one or more processors, further cause the one or more processors to perform a step comprising:
generating a map, the map including a plurality of cotton cluster indicators, each cotton cluster indicator of the plurality of cotton cluster indicators corresponding to one of the plurality of cotton clusters, located at a location of the map corresponding to a location of the corresponding cotton cluster in a worksite, and visually distinguished to indicate the cotton grouping to which the corresponding cotton cluster belongs.

7. The cotton harvesting system of claim 6, wherein the map includes a plurality of harvest area indicators, each harvest area indicator of the plurality of harvest area indicators corresponding to one of the plurality of cotton clusters, located at an area of the map corresponding to an area of the worksite from which the cotton, of the corresponding cotton cluster, was harvested.

8. The cotton harvesting system of claim 6, wherein the map includes a plurality of characteristic indicators, each characteristic indicator indicating a value of a characteristic, different than each of the plurality of cotton quality characteristics, located at an area of the map corresponding to an area of the worksite to which the value of the characteristic corresponds.

9. The cotton harvesting system of claim 6, wherein the computer executable instructions, when executed by the one or more processors, further cause the one or more processors to perform a step comprising:
generating a display, the display including the map and a cotton quality display portion, the cotton quality display portion configured to display the respective set of cotton quality metrics for at least one cotton cluster of the plurality of cotton clusters generated by the cotton harvester.

10. The cotton harvesting system of claim 4, wherein the computer executable instructions, when executed by the one or more processors, further cause the one or more processors to perform a step comprising:
generating a machine assignment assigning a cotton collection and transport machine to a cotton grouping of the plurality of cotton groupings.

11. The cotton harvesting system of claim 10, wherein the computer executable instructions, when executed by the one or more processors, further cause the one or more processors to perform a step comprising:
generating a route for the cotton collection and transport machine based on the machine assignment.

12. The cotton harvesting system of claim 1, wherein the control signal controls a controllable subsystem of the cotton harvester.

13. The cotton harvesting system of claim 1, wherein the control signal controls a controllable subsystem of a cotton collection and transport machine.

14. A computer implemented method comprising:
detecting, with one or more cotton quality characteristic sensors on-board a cotton harvester, a plurality of cotton quality characteristics and generating cotton quality characteristic sensor data indicative of the detected plurality of cotton quality characteristics;
obtaining the cotton quality characteristic sensor data;
determining, for each cotton cluster of a plurality of cotton clusters generated by the cotton harvester, a respective set of cotton quality metrics based on the cotton quality characteristic sensor data, wherein, for each respective set, each cotton quality metric corresponds to a different cotton quality characteristic of the plurality of cotton quality characteristics than the other cotton quality metrics of the respective set; and
generating one or more control signals based on at least one respective set of cotton quality metrics.

15. The computer implemented method of claim 14 and further comprising:
providing the respective set of cotton quality metrics for each of cotton cluster of the plurality of cotton clusters generated by the cotton harvester to a cotton grouping model to obtain, as a cotton grouping model output, a plurality of cotton groupings, each cotton grouping, of the plurality of groupings, comprising a different set of cotton clusters of the plurality of cotton clusters generated by the cotton harvester.

16. The computer implemented method of claim 15 and further comprising:
generating a map, the map including a plurality of cotton cluster indicators, each cotton cluster indicator of the plurality of cotton cluster indicators corresponding to one of the plurality of cotton clusters, located at a location of the map corresponding to a location of the corresponding cotton cluster in a worksite, and visually distinguished to indicate the cotton grouping to which the corresponding cotton cluster belongs.

17. The computer implemented method of claim 16 and further comprising:
generating a machine assignment assigning a cotton collection and transport machine to a cotton grouping of the plurality of cotton groupings; and
generating a route for the cotton collection and transport machine based on the machine assignment.

18. The computer implemented method of claim 14, wherein generating the one or more control signals comprises one or more of:
generating a control signal to control a controllable subsystem of the cotton harvester; or
generating a control signal to control a controllable subsystem of cotton collection and transport machine.

19. A cotton harvester comprising:
one or more cotton quality characteristic sensors configured to detect a plurality of cotton quality characteristics and to generate cotton quality characteristic sensor data indicative of the detected plurality of cotton quality characteristics;
one or more processors; and
memory storing computer executable instructions, executable by the one or more processors, the computer executable instructions, when executed by the one or more processors, causing the one or more processors perform steps comprising:
obtaining the cotton quality characteristic sensor data;
determining, for each cotton cluster, of a plurality of cotton clusters generated by the cotton harvester, a respective set of cotton quality metrics based on the cotton quality characteristic sensor data, wherein, for each respective set, each cotton quality metric corresponds to a different cotton quality characteristic of the plurality of cotton quality characteristics than the other cotton quality metrics of the respective set; and
generating a control signal based on at least one respective set of cotton quality metrics.

20. The cotton harvester of claim 19, wherein the computer executable instructions, when executed by the one or more processors, further cause the one or more processors to perform a step comprising:
providing the respective set of cotton quality metrics for each cotton cluster of the plurality of cotton clusters generated by the cotton harvester to a cotton grouping model to obtain, as a cotton grouping model output, a plurality of cotton groupings, each cotton grouping, of the plurality of cotton groupings, comprising a different set of cotton clusters of the plurality of cotton clusters generated by the cotton harvester.
